(19) United States
(12) Patent Application Publication
(10) Pub. No.: US 2025/0031650 A1
(43) Pub. Date: Jan. 30, 2025

(54) WHEAT PLANTS WITH REDUCED FREE ASPARAGINE CONCENTRATION IN GRAIN

(71) Applicant: Colorado State University Research Foundation, Fort Collins, CO (US)

(72) Inventors: Stephen Pearce, Harpenden (GB); Caitlynd Krosch, Fridley, MN (US); Andrew Katz, Fort Collins, CO (US); Rocio Alarcon Reverte, Harpenden (GB); Karl Ravet, Fort Collins, CO (US); Forrest Wold-McGimsey, Fort Collins, CO (US)

(21) Appl. No.: 18/707,052

(22) PCT Filed: Nov. 4, 2022
(86) PCT No.: PCT/US2022/079297

§ 371 Date: May 2, 2024

Related U.S. Application Data
(60) Provisional application No. 63/276,405, filed on Nov. 5, 2021.

(51) Int. Cl. A01H 6/46; C12N 5/04; C12N 9/00; C12N 15/82; C12Q 1/6895

(52) U.S. Cl. CPC A01H 6/4678; C12N 5/04; C12N 9/93; C12N 15/8251; C12Q 1/6895; C12Y 603/05004; C12N 2510/00; C12Q 2600/13

(57) ABSTRACT

The disclosure relates to compositions and methods for reducing free asparagine concentration in the grain of wheat plants. The compositions comprise wheat plants, plant parts, plant cells, and seeds comprising a loss-of-function mutation in at least one endogenous ASPARAGINE SYNTHETASE 2 (ASN2) gene. The grain of wheat plants has reduced acrylamide-forming potential. Low acrylamide products produced from the grain are also provided.

### CROSS REFERENCE TO RELATED APPLICATIONS

This application claims priority to provisional application U.S. Ser. No. 63/276,405, filed Nov. 5, 2021, which is incorporated herein by reference in its entirety.

### SEQUENCE LISTING XML

The instant application contains a sequence listing, which has been submitted in XML file format by electronic submission and is hereby incorporated by reference in its entirety. Said XML file, created on Nov. 2, 2022, is named P13995WO00.xml and is 124,471 bytes in size.

### TECHNICAL FIELD

The present disclosure relates to the field of biotechnology. More specifically, the present disclosure relates to compositions and methods for producing wheat plants with reduced free asparagine concentration in their grain.

### BACKGROUND

Acrylamide is a potent neurotoxin and causes cancer in rodents. For humans, acrylamide is classified as a probable carcinogen by the International Agency for Research on Cancer (IARC). There is a strong and growing incentive to reduce potential sources of acrylamide exposure in humans, and regulatory agencies are beginning to specify limits in recognition of this threat. A major source of acrylamide exposure is the consumption of processed foods that are rich in carbohydrates. Acrylamide levels are highest in potatoes and coffee, but wheat (Triticum aestivum L.) is also a major source of dietary acrylamide due to the high volume of bread products consumed in the human diet. Acrylamide is a processing contaminant that accumulates in the high temperature and low moisture conditions during baking as a product of the Maillard reaction. Modifying production conditions such as baking at lower temperatures or adding chemical amendments can reduce the level of acrylamide formation, but these are often impractical to implement. Thus, there is need in the art for other approaches to reduce dietary exposure to acrylamide.

### SUMMARY

As asparagine provides the carbon skeleton on which acrylamide is formed, free asparagine concentration is the limiting substrate for acrylamide formation. In wheat, free asparagine concentrations in the grain are highly correlated with acrylamide levels in baked products. Therefore, one approach to reduce dietary exposure to acrylamide is to develop wheat plants with reduced free asparagine concentration in their grain.

Modified wheat plants, and progeny thereof, comprising a loss-of-function mutation in at least one endogenous ASPARAGINE SYNTHETASE 2 (ASN2) gene selected from ASN-A2, ASN-B2, and ASN-D2 that confers reduced free asparagine concentration in the grain of the wheat plant are provided. In certain embodiments, the plant comprises a loss-of-function mutation in the ASN-A2 gene and the ASN-B2 gene, the ASN-A2 gene and the ASN-D2 gene, or the ASN-B2 gene and the ASN-D2 gene. In certain other embodiments, the plant comprises a loss-of-function mutation in each of ASN-A2, ASN-B2, and ASN-D2.

Plant parts, plant cells, and seeds of the modified wheat plants are also provided.

Methods of reducing free asparagine concentration in the grain of a wheat plant are provided. The methods comprise introducing a loss-of-function mutation in at least one endogenous ASN2 gene selected from ASN-A2, ASN-B2, and ASN-D2. In certain embodiments, the mutation is introduced by genome editing.

Methods of producing a wheat plant having grain with reduced free asparagine concentration are provided. The methods comprising (a) crossing a plant of the disclosure with itself or another plant to produce seed; and (b) growing a progeny plant from the seed to produce a plant having grain with reduced free asparagine concentration. In certain embodiments, the methods further comprise (c) crossing the progeny plant with itself or another plant; and (d) repeating steps (b) and (c) for an additional 0-7 generations to produce a plant having grain with reduced free asparagine concentration.

A crop comprising a plurality of the wheat plants of the disclosure planted together in an agricultural field is provided.

Commodity plant products prepared from the plants of the disclosure or parts thereof are provided. In certain embodiments, the product is grain, flour, a baked good, cereal, pasta, a beverage, livestock feed, biofuel, straw, a construction material, or starch. In certain embodiments, the products are low acrylamide products. Methods for producing the commodity plant products are also provided.

Primers and primer pairs for determining the presence of a loss-of-function mutation in an ASN-A2 gene, an ASN-B2 gene, or an ASN-D2 gene are provided. Methods of using the primers and primer pairs are also provided. The methods comprise assaying a nucleic acid sample from a wheat plant with at least one primer or primer pair of the disclosure.

A guide RNA for editing ASN2 genes is provided. The guide RNA sequence shares 100% sequence identity in all ASN2 genes present in wheat varieties with assembled genomes and can be applied broadly in diverse germplasm with very few off-target effects. Vectors encoding the guide RNA are provided. Wheat plant cells comprising a Cas9 nuclease and the guide RNA are also provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A-C shows characterization of the ASN-A2 mutant. FIG. 1A shows details of the induced mutations in each line. Mutation position represents the mutated residue in the wild-type ASN-A2 coding sequence. Protein mutations refer to the amino acid mutated where W=Tryptophan and *=stop codon. FIG. 1B is a schematic representation of the selected mutations in each line and their effects on protein translation. Glutamine amidotransferase (GATase) and ASN synthase conserved domains are indicated and drawn to scale based on protein size. FIG. 1C shows transcript levels of ASN2 genes in grain tissues of wild-type and mutant lines at 21 days after anthesis (DAA) and 28 DAA. Error bars represent standard error of the mean (n=6, except for T4-2032 wild-type samples, where n=5). Student t-tests were performed for each gene between genotypes at each timepoint, *=P<0.05; **=P<0.01; *** P<0.001.

FIG. 2A-C shows the genotyping assays to detect the mutations in the ASN-A2 gene. FIG. 2A shows the position of the three primers in ASN-A2 target gene to detect the G468A mutation in line T4-1388 (SEQ ID NO: 60-63). FIG. 2B shows the position of the three primers in ASN-A2 target gene to detect the G446A mutation in line T4-2032 (SEQ ID NOs: 64-67). FIG. 2C shows the CAPS marker (SEQ ID NOs: 14 and 15) to detect mutation G585A in line T6-1048. PCR products were digested with StyI. Template DNA from plants carrying the A residue at this position (Mutant type, MT) were not digested and present as a 1,063 bp product. Template DNA from plants carrying the G residue at this position (Wildtype, WT) were digested into two products, 946 bp and 117 bp (not visible on this gel). Heterozygous plants exhibit a mix of both products (Het).

FIG. 3A-B shows free asparagine concentration in mature grain of wild-type and mutant asn-a2 sister lines in BC₁F₂ materials grown in 2019 (FIG. 3A) and BC₂F₂ materials grown in 2020 (FIG. 3B). *=P<0.05; *** P<0.001.

FIG. 4A-B shows mean free asparagine concentrations (±standard error) in elite winter wheat cultivars grown in field trials grouped by environment (FIG. 4A) or grouped by variety (FIG. 4B). Different letters indicate significant pairwise differences, calculated by Tukey's post-hoc test (P<0.05).

FIG. 5 shows sgRNA design to target three ASN2 genes. The 20-nucleotide target sequence and three nucleotide protospacer adjacent motif (PAM) are indicated (SEQ ID NOs: 68-79). Gene sequences are displayed in 5′-3′ orientation, and the sgRNA is designed on the antisense strand. The targeted region is in the first exon, within the sequence encoding the conserved GA Tase domain and upstream of the ASN synthetase domain.

FIG. 6 shows wild-type and edited alleles in a transformed ‘CO18D181R’ individual (SEQ ID NOs: 80-83). sgRNA and PAM sequences are indicated. All mutations in ASN-A2 and ASN-D) 2 are homozygous while mutations in ASN-B2 are heterozygous (A/G) for the two alleles indicated.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “a mutation” includes a single mutation, as well as two or more mutations; reference to “a plant” includes one plant, as well as two or more plants; and so forth. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the terms “include,” “includes,” and “including” are to be construed as at least having the features to which they refer while not excluding any additional unspecified features.

Asparagine synthetase (ASN) enzymes catalyze the Adenosine Triphosphate (ATP)-dependent assimilation of inorganic nitrogen in the form of ammonium into asparagine. The wheat genome contains three homologous copies of five asparagine synthetase genes that exhibit distinct expression profiles during development. The ASN2 genes are notable for their grain-specific expression profile.

The present disclosure relates to modified wheat plants comprising a loss-of-function mutation in at least one endogenous ASN2 gene selected from ASN-A2, ASN-B2, and ASN-D2. The loss of function mutation in one, two, or in each of ASN-A2, ASN-B2, and ASN-D2 may be a deletion, insertion, or substitution with reference to the wild type ASN-A2, ASN-B2, and ASN-D2 sequence. In certain embodiments, the wild type ASN-A2 comprises or consists of SEQ ID NO: 1, or an allelic variant thereof. The corresponding amino acid sequence is SEQ ID NO: 7. In certain embodiments, the wild type ASN-B2 comprises or consists of SEQ ID NO: 2, or an allelic variant thereof. The corresponding amino acid sequence is SEQ ID NO: 8. In certain embodiments, the wild type ASN-D2 comprises or consists of SEQ ID NO: 3, or an allelic variant thereof. The corresponding amino acid sequence is SEQ ID NO: 9. TABLE 1 provides a summary of the wild type ASN-A2, ASN-B2, and ASN-D2 sequences.

TABLE 1

| ASN2 Gene | Genomic sequence | cDNA sequence | Protein sequence |
|---|---|---|---|
| ASN-A2 | SEQ ID NO: 1 | SEQ ID NO: 4 | SEQ ID NO: 7 |
| ASN-B2 | SEQ ID NO: 2 | SEQ ID NO: 5 | SEQ ID NO: 8 |
| ASN-D2 | SEQ ID NO: 3 | SEQ ID NO: 6 | SEQ ID NO: 9 |

The ASN2 genes as defined above include any regulatory sequences that are 5′ or 3′ of the transcribed region, including the promoter region, that regulate the expression of the associated transcribed region, and introns within the transcribed regions.

The phrase “allelic variant” as used herein refers to a polynucleotide sequence variant that occurs in a different strain, variety, or isolate of a given organism. It would be understood that there is natural variation in the sequences of ASN2 genes from different wheat varieties. The allelic variants are readily recognizable by the skilled artisan on the basis of genome synteny and sequence similarity.

An allele is a variant of a gene at a single genetic locus. A diploid organism has two sets of chromosomes. Each chromosome has one copy of each gene (one allele). If both alleles are the same the organism is homozygous with respect to that gene, if the alleles are different, the organism is heterozygous with respect to that gene. The interaction between alleles at a locus is generally described as dominant or recessive. A loss of function mutation, which includes a partial loss of function mutation in an allele, means a mutation in the allele leading to no or a reduced level or activity of ASN2 enzyme in the grain. The mutation may mean, for example, that no or less RNA is transcribed from the gene comprising the mutation, that less protein is translated, or that the protein produced has no or reduced activity. Alleles that do not encode or are not capable of leading to the production any active enzyme are null alleles. A “reduced” amount or level of protein means reduced relative to the amount or level produced by the corresponding wild-type allele. A “reduced” activity means reduced relative to the corresponding wild-type ASN2 enzyme. Different alleles may have the same or a different mutation and different alleles may be combined using methods known in the art. In some embodiments, the amount of ASN2 protein is reduced because there is less transcription or translation of the ASN2 gene. In some embodiments, the amount by weight of ASN2 protein is reduced even though there is a wild-type number of ASN2 protein molecules, because some of the proteins produced are shorter than wild-type ASN2 protein, e.g., the mutant ASN2 protein is truncated due to a premature translation termination signal.

The wheat plants of the disclosure can be produced and identified after mutagenesis or genome editing. Mutant wheat plants having a mutation in a single ASN2 gene which can be combined by crossing and selection with other ASN2 mutations to generate the wheat plants of the disclosure can be either synthetic, for example, by performing site-directed mutagenesis on the nucleic acid, or induced by mutagenic treatment, or may be naturally occurring, i.e., isolated from a natural source. Generally, a progenitor plant cell, tissue, seed or plant may be subjected to mutagenesis or genome editing to produce single or multiple mutations, such as nucleotide substitutions, deletions, additions and/or codon modification.

Mutagenesis can be achieved by chemical or radiation means, for example ethyl methanesulfonate (EMS), sodium azide, or gamma irradiation treatment of seed, well known in the art. Chemical mutagenesis tends to favor nucleotide substitutions rather than deletions.

Genome editing methods can produce site-specific mutants in a plant genome. Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption. Engineered nucleases useful in the methods of the present disclosure include zinc finger nucleases (ZFNs), transcription activator-like (TAL) effector nucleases (TALEN) and CRISPR/Cas9 type nucleases.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In embodiments, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis2His2 type of zinc finger that is very generally represented, for example, by the zinc finger transcription factors TFIIIA or Sp1. In embodiments, the zinc finger domain comprises three Cis2His2 type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques (see, for example, Bibikova et al., 2002).

The ZFN DNA-cleavage domain is derived from a class of non-specific DNA cleavage domains, for example the DNA-cleavage domain of a Type II restriction enzyme such as Fold (Kim et al., 1996). Other useful endonucleases may include, for example, HhaI, HindIII, Nod, BbvCI, EcoRI, BglI, and AlwI.

A transcription activator-like (TAL) effector nuclease (TALEN) comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

Distinct from the site-specific nucleases described above, the clustered regulatory interspaced short palindromic repeats (CRISPR)/Cas system provides an alternative to ZFNs and TALENs for inducing targeted genetic alterations, via RNA-guided DNA cleavage.

CRISPR systems rely on CRISPR RNA (crRNA) and transactivating chimeric RNA (tracrRNA) for sequence-specific cleavage of DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components. The Cas endonuclease may be converted into a nickase to provide additional control over the mechanism of DNA repair (Cong et al., 2013).

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

A mutagenized or genome edited population of wheat may be screened directly for the ASN2 genotype or indirectly by screening for a phenotype that results from mutations in the ASN2 gene. Screening directly for the genotype can include assaying for the presence of mutations in the ASN2 gene, which may be observed in PCR assays by the absence of specific ASN2 markers as expected when some of the genes are deleted, or heteroduplex based assays as in TILLING. Screening for the phenotype can comprise screening for a loss or reduction in amount of one or more ASN2 proteins by ELISA or affinity chromatography, or reduced free asparagine concentration in the grain.

Plants and seeds of the disclosure can be produced using the process known as TILLING (Targeting Induced Local Lesions IN Genomes), in that one or more of the mutations in the wheat plants or grain may be produced by this method. In a first step, introduced mutations such as novel single base pair changes are induced in a population of plants by treating seeds or pollen with a chemical or radiation mutagen, and then advancing plants to a generation where mutations will be stably inherited, typically an M2 generation where homozygotes may be identified. DNA is extracted, and seeds are stored from all members of the population to create a resource that can be accessed repeatedly over time. For a TILLING assay, PCR primers are designed to specifically amplify a single gene target of interest. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cell I, that recognizes and cleaves mismatched DNA is the key to discovering novel SNPs within a TILLING population. Alternatively, the members of the population are screened by exome or genome sequencing.

Using this approach, many thousands of plants can be screened to identify any individual with a single base change as well as small insertions or deletions (e.g., 1-30 bp) in any gene or specific region of the genome. Genomic fragments being assayed can range in size anywhere from 0.3 to 1.6 kb. At 8-fold pooling and amplifying 1.4 kb fragments with 96 lanes per assay, this combination allows up to a million base pairs of genomic DNA to be screened per single assay, making TILLING a high-throughput technique. TILLING is further described in Slade and Knauf, 2005, and Henikoff et al., 2004.

Identified mutations may then be introduced into desirable genetic backgrounds by crossing the mutant with a plant of the desired genetic background and performing a suitable number of backcrosses to cross out the originally undesired parent background.

In the context of this disclosure, an “induced mutation” or “introduced mutation” is an artificially induced genetic variation which may be the result of chemical, radiation or biologically-based mutagenesis, for example genome editing. In certain embodiments, the mutations are null mutations such as nonsense mutations, frameshift mutations, deletions, insertional mutations or splice-site variants which completely inactivate the gene. In certain other embodiments, the mutations are partial mutations which retain some ASN2 activity, but less than wild-type levels of the enzyme. Nucleotide insertional derivatives include 5′ and 3′ terminal fusions as well as intra-sequence insertions of single or multiple nucleotides. Insertional nucleotide sequence variants are those in which one or more nucleotides are introduced into a site in the nucleotide sequence, either at a predetermined site as is possible with the CRISPR/Cas system or other genome editing methods, or by random insertion with suitable screening of the resulting product. Deletional variants are characterized by the removal of one or more nucleotides from the sequence. Preferably, a mutant gene has only a single insertion or deletion of a sequence of nucleotides relative to the wild-type gene. The deletion may be extensive enough to include one or more exons or introns, both exons and introns, an intron-exon boundary, a part of the promoter, the translational start site, or even the entire gene. Insertions or deletions within the exons of the protein coding region of a gene which insert or delete a number of nucleotides which is not an exact multiple of three, thereby causing a change in the reading frame during translation, almost always abolish activity of the mutant gene comprising such insertion or deletion.

Substitutional nucleotide variants are those in which at least one nucleotide in the sequence has been removed and a different nucleotide inserted in its place. The preferred number of nucleotides affected by substitutions in a mutant gene relative to the wild-type gene is a maximum of ten nucleotides, more preferably a maximum of 9, 8, 7, 6, 5, 4, 3, or 2, or most preferably only one nucleotide. Substitutions may be “silent” in that the substitution does not change the amino acid defined by the codon. Nucleotide substitutions may reduce the translation efficiency and thereby reduce the ASN2 expression level, for example by reducing the mRNA stability or, if near an exon-intron splice boundary, alter the splicing efficiency. Silent substitutions that do not alter the translation efficiency of an ASN2 gene are not expected to alter the activity of the genes and are therefore regarded herein as non-mutant, i.e. such genes are active variants and not encompassed in “mutant alleles”. Alternatively, the nucleotide substitution(s) may change the encoded amino acid sequence and thereby alter the activity of the encoded enzyme, particularly if conserved amino acids are substituted for another amino acid which is quite different i.e. a non-conservative substitution.

A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif. Conservative substitution tables are well known in the art (see for example Creighton (1984) Proteins. W.H. Freeman and Company (Eds).

The term “mutation” as used herein does not include silent nucleotide substitutions which do not affect the activity of the gene, and therefore includes only alterations in the gene sequence which affect the gene activity. The term “polymorphism” refers to any change in the nucleotide sequence including such silent nucleotide substitutions. Screening methods may first involve screening for polymorphisms and secondly for mutations within a group of polymorphic variants.

As is understood in the art, hexaploid wheats such as bread wheat comprise three genomes which are commonly designated the A, B and D genomes, while tetraploid wheats such as durum wheat comprise two genomes commonly designated the A and B genomes. Each genome comprises 7 pairs of chromosomes which may be observed by cytological methods during meiosis and thus identified, as is well known in the art.

The terms “plant(s)” and “wheat plant(s)” as used herein as a noun generally refer to whole plants, but when “plant” or “wheat” is used as an adjective, the terms refer to any substance which is present in, obtained from, derived from, or related to a plant or a wheat plant, such as for example, plant organs (e.g. leaves, stems, roots, flowers), single cells (e.g. pollen), seeds, plant cells including for example tissue cultured cells, products produced from the plant such as “wheat flour”, “wheat grain”, and the like. Plantlets and germinated seeds from which roots and shoots have emerged are also included within the meaning of “plant”. The term “plant parts” as used herein refers to one or more plant tissues or organs which are obtained from a whole plant. Plant parts include vegetative structures (for example, leaves, stems), roots, floral organs/structures, seed (including embryo, endosperm, and seed coat), plant tissue (for example, vascular tissue, ground tissue, and the like), cells and progeny of the same. A progeny plant can be from any filial generation, e.g., F1, F2, F3, F4, F5, F6, F7, etc. The term “plant cell” as used herein refers to a cell obtained from a plant or in a plant, and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, pollen, and various forms of aggregations of plant cells in culture, such as calli. Plant tissues in or from seeds such as wheat seeds are seed coat, endosperm, scutellum, aleurone layer and embryo.

In some embodiments, plant cells of the present disclosure are capable of regenerating a plant or plant part. In other embodiments, plant cells are not capable of regenerating a plant or plant part. Examples of cells not capable of regenerating a plant include, but are not limited to, endosperm, seed coat (testa and pericarp), and root cap.

Cereals as used herein means plants or grain of the monocotyledonous families Poaceae or Graminae which are cultivated for the edible components of their seeds, and includes wheat, barley, maize, oats, rye, rice, sorghum, triticale, millet, and buckwheat. In certain embodiments, the plant or grain is a Triticeae plant or grain (e.g., wheat, barley, or rye). In certain embodiments, the plant or grain is a wheat plant or grain.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye Secale cereale, including but not limited to Triticale. In certain embodiments, the wheat plant is suitable for commercial production of grain, such as commercial varieties of hexaploid wheat or durum wheat, having suitable agronomic characteristics which are known to those skilled in the art. In certain embodiments, the wheat is Triticum aestivum ssp. aestivum or Triticum turgidum ssp. durum.

As used herein, the term “barley” refers to any species of the genus Hordeum, including progenitors thereof, as well as progeny thereof produced by crosses with other species. In certain embodiments, the plant is of a Hordeum species which is commercially cultivated such as, for example, a strain or cultivar or variety of Hordeum vulgare or suitable for commercial production of grain.

As used herein, the term “rye” refers to any species of the genus Secale, including progenitors thereof, as well as progeny thereof produced by crosses with other species. In certain embodiments, the plant is of a Secale species which is commercially cultivated such as, for example, a strain or cultivar or variety of Secale cereale or suitable for commercial production of grain.

The wheat plants of the disclosure may be crossed with plants containing a more desirable genetic background, and therefore the disclosure includes the transfer of the low asparagine trait to other genetic backgrounds. After the initial crossing, a suitable number of backcrosses may be carried out to remove a less desirable background. ASN2 allele-specific PCR-based markers such as those described herein may be used to screen for or identify progeny plants or grain with the desired combination of alleles, thereby tracking the presence of the alleles in the breeding program. The desired genetic background may include a suitable combination of genes providing commercial yield and other characteristics such as agronomic performance or abiotic stress resistance. The genetic background may comprise one or more transgenes such as, for example, a gene that confers tolerance to a herbicide such as glyphosate. The desired genetic background of the wheat plant will include considerations of agronomic yield and other characteristics. Such characteristics might include whether it is desired to have a winter or spring types, growth habit, agronomic performance, disease resistance and abiotic stress resistance.

Marker assisted selection is a well-recognized method of selecting for heterozygous plants obtained when backcrossing with a recurrent parent in a classical breeding program. The population of plants in each backcross generation will be heterozygous for the gene(s) of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants.

Procedures such as crossing wheat plants, self-fertilizing wheat plants or marker-assisted selection are standard procedures and well known in the art. Transferring alleles from tetraploid wheat such as durum wheat to a hexaploid wheat, or other forms of hybridization, is also known in the art.

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity, and plant height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

To identify the desired phenotypic characteristic, wheat plants that contain a mutant ASN2 allele or other desired genes are typically compared to control plants. When evaluating a phenotypic characteristic associated with enzyme activity such as asparagine content in the grain, the plants to be tested and control plants are grown under growth chamber, greenhouse, open top chamber and/or field conditions. Identification of a particular phenotypic trait and comparison to controls is based on routine statistical analysis and scoring. Statistical differences between plants lines can be assessed by comparing, for example, enzyme activity between plant lines within each tissue type expressing the enzyme.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one embodiment, a modified plant provided herein comprises no non-plant genetic material or sequences. In yet another embodiment, a modified plant provided herein comprises no interspecies genetic material or sequences.

In certain embodiments, the wheat plants, wheat plant parts and products therefrom of the disclosure are non-transgenic for genes that inhibit expression of ASN2 (e.g., they do not comprise a transgene encoding an RNA molecule that reduces expression of the endogenous ASN2 genes). In certain embodiments, they may comprise other transgenes, e.g., herbicide tolerance genes. In certain embodiments, the wheat plant, grain and products therefrom are non-transgenic, i.e. they do not contain any transgene.

The terms “transgenic plant” and “transgenic wheat plant” as used herein refer to a plant that contains a genetic construct (“transgene”) not found in a wild-type plant of the same species, variety or cultivar. That is, transgenic plants (transformed plants) contain genetic material that they did not contain prior to the transformation. A “transgene” as referred to herein has the normal meaning in the art of biotechnology and refers to a genetic sequence which has been produced or altered by recombinant DNA or RNA technology and which has been introduced into the plant cell. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. A “non-transgenic plant” is one which has not been genetically modified by the introduction of genetic material by recombinant DNA techniques.

Any of several methods may be employed to determine the presence of a transgene in a transformed plant. For example, polymerase chain reaction (PCR) may be used to amplify sequences that are unique to the transformed plant, with detection of the amplified products by gel electrophoresis or other methods. DNA may be extracted from the plants using conventional methods and the PCR reaction carried out using primers that will distinguish the transformed and non-transformed plants. An alternative method to confirm a positive transformant is by Southern blot hybridization, well known in the art. Wheat plants which are transformed may also be identified i.e. distinguished from non-transformed or wild-type wheat plants by their phenotype, for example conferred by the presence of a selectable marker gene, or by immunoassays that detect or quantify the expression of an enzyme encoded by the transgene, or any other phenotype conferred by the transgene.

In certain embodiments, biological samples from the plants of the disclosure are provided. As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, flakes, syrup, oil, starch, and cereals manufactured in whole or in part to contain crop plant by-products. In certain embodiments, the biological sample is “non-regenerable” (i.e., incapable of being regenerated into a plant or plant part).

Several embodiments provide a commodity plant product prepared from the plants of the disclosure. The plants of the present disclosure may be grown or harvested for grain, primarily for use as food for human consumption or as animal feed, or for fermentation or industrial feedstock production such as ethanol production, among other uses. Alternatively, the wheat plants may be used directly as feed. The plants of the present disclosure may be useful for food production and in particular for commercial food production. Such food production might include the making of flour, dough, semolina or other products from the grain that might be an ingredient in commercial food production.

The product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g. by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally, the plants are grown for some time before the product is produced.

Wheat may be used to produce a variety of products, including, but not limited to, grain, flour, baked goods, cereals, crackers, pasta, beverages, livestock feed, biofuel, straw, construction materials, and starches. The hard wheat classes are milled into flour used for breads, while the soft wheat classes are milled into flour used for pastries and crackers. Wheat starch is used in the food and paper industries as laundry starches, among other products.

The disclosure thus provides flour, meal or other products produced from the low asparagine wheat grain. These may be unprocessed or processed, for example by fractionation or bleaching, or heat treated to stabilize the product such as flour. The disclosure includes methods of producing flour, meal, starch granules, or starch from the grain or from an intermediate product such as flour. Such methods include, for example, milling, grinding, rolling, flaking or cracking the grain.

The present disclosure also extends to wheat flour, such as wholemeal wheat flour, or other processed products obtained from the grain such as semolina, isolated wheat starch granules, isolated wheat starch or wheat bran produced from the grain of the disclosure. The β-glucan content of the wholemeal flour is essentially the same as for the wheat grain, as described above. In an embodiment, the flour is wheat endosperm flour (white flour). The white flour has a lower bran content than the wholemeal flour from which it is obtained. The flour or bran may have been stabilized by heat treatment.

The present disclosure also provides a food ingredient that comprises the grain or flour. In certain embodiments, the food or drink ingredient is packaged ready for sale. The food or drink ingredient may be incorporated into a mixture with another food or drink ingredient, such as, for example, a cake mix, a pancake mix or a dough. The food ingredient may be used in a food product at a level of at least 1%, preferably at least 10%, on a dry weight basis, and the drink ingredient may be used in a drink product at a level of at least 0.1% on a weight basis. If the food product is a breakfast cereal, bread, cake or other farinaceous product, higher incorporation rates are preferred, such as at a level of at least 20% or at least 30%. Up to 100% of the ingredient (grain, flour such as wholemeal flour etc.) in the food product may be an ingredient of the disclosure.

The grain of the present disclosure and the ingredients obtained therefrom may be blended with essentially wild-type grain or other ingredients. The disclosure therefore provides a composition comprising traditional wheat grain or an ingredient obtained therefrom in addition to the wheat grain of the disclosure, or an ingredient obtained therefrom. In such compositions, it is preferred that the grain of the present disclosure and/or the ingredient obtained therefrom comprises at least 10% by weight of the composition. The traditional wheat grain ingredient may be, for example, flour such as wholemeal flour, semolina, a starch-containing ingredient, purified starch or bran.

The grain of the present disclosure may also be milled to produce a milled wheat product. This will typically involve obtaining wheat grain, milling the grain to produce flour, and optionally, separating any bran from the flour. Milling the grain may be by dry milling or wet milling. The grain may be conditioned to having a desirable moisture content prior to milling, preferably about 10% or about 14% on a weight basis, or the milled product such as flour or bran may be processed by treatment with heat to stabilize the milled product. As will be understood, the free asparagine concentration of the milled product corresponds to the free asparagine concentration in the wheat grain or the component of the wheat grain which is represented in the milled product.

The present disclosure also provides a method of producing a product, wherein the method comprises processing a wheat plant of the disclosure to obtain the product.

In certain embodiments, the method comprises (i) obtaining or producing a wheat grain of the present disclosure, or flour therefrom, and (ii) processing the wheat grain or flour therefrom to produce the product.

In certain embodiments, the whole grain flour, the coarse fraction, or the refined flour may be a component (ingredient) of a food product and may be used to produce a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, chapatti, roti, naan, animal food or pet food.

As used herein, the term “grain” generally refers to mature, harvested seed of a plant but can also refer to grain after imbibition or germination, according to the context. Mature cereal grain such as wheat commonly has a moisture content of less than about 18-20%. As used herein, the term “seed” includes harvested seed but also includes seed which is developing in the plant post anthesis and mature seed comprised in the plant prior to harvest.

In certain embodiments, the free asparagine concentration of the grain is reduced by at least 10%, at least 20%, at least 30%, at least 40%, at least 50%, at least 60%, at least 70%, at least 80%, at least 90%, or at least 95% relative to that of corresponding wild-type grain. In certain embodiments, the free asparagine concentration of the grain is reduced by about 10% to about 90%, about 20% to about 80%, or about 30% to about 70% relative to that of corresponding wild-type grain.

As used herein, “germination” refers to the emergence of the root tip from the seed coat after imbibition. “Germination rate” refers to the percentage of seeds in a population which have germinated over a period of time, for example 7 or 10 days, after imbibition. Germination rates can be calculated using techniques known in the art. For example, a population of seeds can be assessed daily over several days to determine the germination percentage over time. With regard to grain of the present disclosure, as used herein the term “germination rate which is substantially the same” means that the germination rate of the grain is at least 90% that of corresponding wild-type grain. In certain embodiments, the grain of the present disclosure has a germination rate of about 70% to about 100% relative to that of corresponding wild-type grain. In certain embodiments, the grain of the present disclosure has a germination rate of at least about 70%, at least about 80%, at least about 90%, at least about 95%, or at least about 99% relative to that of corresponding wild-type grain.

The terms “polypeptide” and “protein” are generally used interchangeably herein. The terms “proteins” and “polypeptides” as used herein also include variants, mutants, modifications and/or derivatives of the polypeptides of the disclosure as described herein. As used herein, “substantially purified polypeptide” refers to a polypeptide that has been separated from the lipids, nucleic acids, other peptides and other molecules with which it is associated in its native state. In certain embodiments, the substantially purified polypeptide is at least 60% free, at least 75% free, or at least 90% free from other components with which it is naturally associated. By “recombinant polypeptide” is meant a polypeptide made using recombinant techniques, i.e., through the expression of a recombinant polynucleotide in a cell.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Methods of alignment of sequences for comparison are well known in the art and can be accomplished using mathematical algorithms such as the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; and the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

Amino acid sequence mutants of the polypeptides of the present disclosure can be prepared by introducing appropriate nucleotide changes into a nucleic acid of the present disclosure or by mutagenesis in vivo such as by chemical or radiation treatment. Such mutants include, for example, deletions, insertions or substitutions of residues within the amino acid sequence. Amino acid sequence deletions generally range from about 1 to 15 residues, about 1 to 10 residues, or about 1 to 5 contiguous residues. Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical i.e., conserved amino acids. These positions may be important for biological activity. Non-conservative substitutions in an ASN2 polypeptide are expected to reduce the activity of the enzyme and many may correspond to an ASN2 polypeptide encoded by a partial loss of function mutant.

As used herein, the term “gene” includes any deoxyribonucleotide sequence which includes a protein coding region or which is transcribed in a cell but not translated, together with associated non-coding and regulatory regions. Such associated regions are typically located adjacent to the coding region on both the 5′ and 3′ ends for a distance of about 2 kb on either side. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. The term “gene” includes synthetic or fusion molecules encoding the proteins of the disclosure described herein. An “endogenous gene” refers to a native gene in its natural location in the genome of an organism.

A genomic form or clone of a gene containing the coding region may be interrupted with non-coding sequences termed “introns” or “intervening regions” or “intervening sequences.” An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

By “operably linked” or “operably associated,” it is meant that the indicated elements are functionally related to each other, and are also generally physically related. Thus, the term “operably linked” or “operably associated” as used herein, refers to nucleotide sequences on a single nucleic acid molecule that are functionally associated. Therefore, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter affects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

The present disclosure refers to various polynucleotides. As used herein, a “polynucleotide” or “nucleic acid” or “nucleic acid molecule” means a polymer of nucleotides, which may be DNA or RNA or a combination thereof, for example a heteroduplex of DNA and RNA, and includes for example mRNA, CRNA, cDNA, RNA, siRNA, shRNA, hpRNA, and single or double-stranded DNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein. In embodiments, the polynucleotide is solely DNA or solely RNA as occurs in a cell, and some bases may be methylated or otherwise modified as occurs in a wheat cell. The polymer may be single-stranded, essentially double-stranded or partly double-stranded. An example of a partly-double stranded RNA molecule is a hairpin RNA (hpRNA), short hairpin RNA (shRNA) or self-complementary RNA which include a double stranded stem formed by basepairing between a nucleotide sequence and its complement and a loop sequence which covalently joins the nucleotide sequence and its complement. Basepairing as used herein refers to standard basepairing between nucleotides, including G: U basepairs in an RNA molecule. “Complementary” means two polynucleotides are capable of basepairing along part of their lengths, or along the full length of one or both.

By “isolated” is meant material that is substantially or essentially free from components that normally accompany it in its native state. As used herein, an “isolated polynucleotide” or “isolated nucleic acid molecule” means a polynucleotide which is at least partially separated from, preferably substantially or essentially free of, the polynucleotide sequences of the same type with which it is associated or linked in its native state. For example, an “isolated polynucleotide” includes a polynucleotide which has been purified or separated from the sequences which flank it in a naturally occurring state, e.g., a DNA fragment which has been removed from the sequences which are normally adjacent to the fragment. In certain embodiments, the isolated polynucleotide is also at least 90% free from other components such as proteins, carbohydrates, lipids etc. The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably connected to the nucleotide sequence to be transcribed in the cell.

The present disclosure refers to use of oligonucleotides which may be used as “probes” or “primers”. The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

As used herein, “oligonucleotides” are polynucleotides up to 50 nucleotides in length. They can be RNA, DNA, or combinations or derivatives of either. Oligonucleotides are typically relatively short single stranded molecules of 10 to 30 nucleotides, commonly 15-25 nucleotides in length, typically comprised of 10-30 or 15-25 nucleotides which are identical to, or complementary to, part of an ASN2 gene or cDNA corresponding to an ASN2 gene. When used as a probe or as a primer in an amplification reaction, the minimum size of such an oligonucleotide is the size required for the formation of a stable hybrid between the oligonucleotide and a complementary sequence on a target nucleic acid molecule. In certain embodiments, the oligonucleotides are at least 15 nucleotides, at least 18 nucleotides, at least 19 nucleotides, at least 20 nucleotides, or at least 25 nucleotides in length. Polynucleotides used as a probe are typically conjugated with a detectable label such as a radioisotope, an enzyme, biotin, a fluorescent molecule, or a chemiluminescent molecule. Oligonucleotides and probes of the disclosure are useful in methods of detecting an allele of an ASN2 gene or other gene associated with a trait of interest, for example reduced free asparagine. Such methods employ nucleic acid hybridization and, in many instances, include oligonucleotide primer extension by a suitable polymerase, for example as used in PCR for detection or identification of wild-type or mutant alleles. In certain embodiments, the oligonucleotides and probes hybridize to an ASN2 gene sequence from wheat, including any of the sequences disclosed herein. In certain embodiments, the oligonucleotide pairs span one or more introns, or a part of an intron and therefore may be used to amplify an intron sequence in a PCR reaction.

The terms “polynucleotide variant” and “variant” and the like refer to polynucleotides displaying substantial sequence identity with a reference polynucleotide sequence and which are able to function in an analogous manner to, or with the same activity as, the reference sequence. These terms also encompass polynucleotides that are distinguished from a reference polynucleotide by the addition, deletion or substitution of at least one nucleotide, or that have, when compared to naturally occurring molecules, one or more mutations. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Accordingly, these terms encompass polynucleotides that encode polypeptides that exhibit enzymatic or other regulatory activity, or polynucleotides capable of serving as selective probes or other hybridizing agents. The terms “polynucleotide variant” and “variant” also include naturally occurring allelic variants. Mutants can be either naturally occurring (that is to say, isolated from a natural source) or synthetic (for example, by performing site-directed mutagenesis on the nucleic acid). In certain embodiments, a polynucleotide variant of the disclosure which encodes a polypeptide with enzyme activity is greater than 400, greater than 500, greater than 600, greater than 700, greater than 800, greater than 900, or greater than 1,000 nucleotides in length, up to the full length of the gene.

A variant of an oligonucleotide of the disclosure includes molecules of varying sizes which are capable of hybridizing, for example, to the wheat genome at a position close to that of the specific oligonucleotide molecules defined herein. For example, variants may comprise additional nucleotides (such as 1, 2, 3, 4, or more), or less nucleotides as long as they still hybridize to the target region. Furthermore, a few nucleotides may be substituted without influencing the ability of the oligonucleotide to hybridize to the target region. In addition, variants may readily be designed which hybridize close (for example, but not limited to, within 50 nucleotides) to the region of the plant genome where the specific oligonucleotides defined herein hybridize.

By “corresponds to” or “corresponding to” in the context of polynucleotides or polypeptides is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein. Terms used to describe sequence relationships between two or more polynucleotides or polypeptides include “reference sequence”, “comparison window”, “sequence identity”, “percentage of sequence identity”, “substantial identity” and “identical”, and are defined with respect to a defined minimum number of nucleotides or amino acid residues or over the full length. The terms “sequence identity” and “identity” are used interchangeably herein to refer to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Nucleotide or amino acid sequences are indicated as “essentially similar” when such sequences have a sequence identity of at least about 95%, particularly at least about 98%, more particularly at least about 98.5%, quite particularly about 99%, especially about 99.5%, more especially about 100%, quite especially are identical. It is clear that when RNA sequences are described as essentially similar to, or have a certain degree of sequence identity with, DNA sequences, thymine (T) in the DNA sequence is considered equal to uracil (U) in the RNA sequence.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In certain embodiments, the present disclosure refers to the stringency of hybridization conditions to define the extent of complementarity of two polynucleotides. “Stringency” as used herein, refers to the temperature and ionic strength conditions, and presence or absence of certain organic solvents, during hybridization. The higher the stringency, the higher will be the degree of complementarity between a target nucleotide sequence and the labelled polynucleotide sequence. “Stringent conditions” refers to temperature and ionic conditions under which only nucleotide sequences having a high frequency of complementary bases will hybridize. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, herein incorporated by reference. Specific hybridization conditions referred to herein are as follows: 1) low stringency hybridization conditions in 6× sodium chloride/sodium citrate (SSC) at about 45° C., followed by two washes in 0.2×SSC, 0.1% SDS at 50-55° C.; 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5 M sodium phosphate, 7% SDS at 65° C., followed by one or more washes at 0.2×SSC, 1% SDS at 65° C.

The present disclosure makes use of vectors for production, manipulation or transfer of genetic constructs. By “vector” is meant a nucleic acid molecule, preferably a DNA molecule derived, for example, from a plasmid, bacteriophage or plant virus, into which a nucleic acid sequence may be inserted. A vector preferably contains one or more unique restriction sites and may be capable of autonomous replication in a defined host cell including a target cell or tissue or a progenitor cell or tissue thereof, or be integrable into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene that can be used for selection of suitable transformants, or sequences that enhance transformation of prokaryotic or eukaryotic (especially wheat) cells such as T-DNA or P-DNA sequences. Examples of such resistance genes and sequences are well known to those of skill in the art.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

A number of techniques are available for the introduction of nucleic acid molecules into a plant cell. The methods do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs.

Accordingly, the nucleotide sequences can be introduced into the cell of interest in a single transformation event, in separate transformation events, or, for example, in plants, as part of a breeding protocol.

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Transformation of a cell may be stable or transient. Thus, in some embodiments, a plant cell is stably transformed with a nucleic acid molecule. In other embodiments, a plant is transiently transformed with a nucleic acid molecule. “Transient transformation” in the context of a polynucleotide means that a polynucleotide is introduced into the cell and does not integrate into the genome of the cell. By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in certain embodiments, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed plants, plant cells and/or plant tissue culture are routine in the art and can be employed in the methods provided herein.

The most commonly used methods to transform wheat plants comprise two steps: the delivery of DNA into regenerable wheat cells and plant regeneration through in vitro tissue culture. Two methods are commonly used to deliver the DNA: T-DNA transfer using Agrobacterium tumefaciens or related bacteria and direct introduction of DNA via particle bombardment, although other methods have been used to integrate DNA sequences into wheat or other cereals. It will be apparent to the skilled person that the particular choice of a transformation system to introduce a nucleic acid construct into plant cells is not essential to or a limitation of the disclosure, provided it achieves an acceptable level of nucleic acid transfer. Such techniques for wheat are well known in the art.

Wheat plants can be produced by introducing a nucleic acid construct into a recipient cell and growing a new plant that comprises and expresses a polynucleotide according to the disclosure. The process of growing a new plant from a transformed cell which is in cell culture is referred to as “regeneration”. Regenerable wheat cells include cells of mature embryos, meristematic tissue such as the mesophyll cells of the leaf base, or from the scutella of immature embryos, obtained 12-20 days post-anthesis, or callus derived from any of these. The most commonly used route to recover regenerated wheat plants is somatic embryogenesis using media such as MS-agar supplemented with an auxin such as 2,4-D and a low level of cytokinin. Any wheat type that is regenerable may be used. Transformation events in one of these more readily regenerable varieties may be transferred to any other wheat cultivars including elite varieties by standard backcrossing. Examples of selectable markers include, but are not limited to, a nucleotide

sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaende that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988). J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in a nucleic acid construct.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in a nucleic acid construct.

### EMBODIMENTS

The following numbered embodiments also form part of the present disclosure:

1. A modified wheat plant, or a progeny thereof, comprising a loss-of-function mutation in at least one endogenous ASPARAGINE SYNTHETASE 2 (ASN2) gene selected from ASN-A2, ASN-B2, and ASN-D2, wherein the mutation confers reduced free asparagine concentration in the grain of the wheat plant.

2. The modified wheat plant of embodiment 1, wherein the plant comprises a loss-of-function mutation in the ASN-A2 gene and the ASN-B2 gene; the ASN-A2 gene and the ASN-D2 gene; or the ASN-B2 gene and the ASN-D2 gene.

3. The modified wheat plant of embodiment 1 or embodiment 2, wherein the plant comprises a loss-of-function mutation in the ASN-A2 gene, the ASN-B2 gene, and the ASN-D2 gene.

4. The modified wheat plant of any one of embodiments 1-3, wherein the mutation introduces a frameshift mutation or a pre-mature stop codon in the ASN2 gene.

5. The modified wheat plant of any one of embodiments 1-4, wherein the mutation is in the first exon encoding the glutamine amidotransferase (GATase) domain of the ASN2 gene.

6. The modified wheat plant of any one of embodiments 1-5, wherein the mutation is an insertion, a deletion, or a substitution of one or more nucleotides from position 172 to 192 with reference to SEQ ID NO: 1, 2, or 3.

7. The modified wheat plant of any one of embodiments 1-6, wherein the free asparagine concentration of the grain is reduced by at least 30%, at least 40%, at least 50%, at least 60%, at least 70%, at least 80%, at least 90%, or at least 95% relative to the free asparagine concentration of grain without the mutation.

8. The modified wheat plant of any one of embodiments 1-7, wherein the grain has a germination rate of about 70% to about 100% relative to the germination rate of grain without the mutation, optionally wherein the grain has a germination rate of at least 90% relative to the germination rate of grain without the mutation.

9. The modified wheat plant of any one of embodiments 1-8, wherein the ASN-A2 gene comprises the nucleotide sequence of SEQ ID NO: 45 or 47, optionally wherein the ASN-A2 gene comprises the nucleotide sequence of SEQ ID NO: 52, 54, or 59.

10. The modified wheat plant of any one of embodiments 1-9, wherein the ASN-B2 gene comprises the nucleotide sequence of SEQ ID NO: 50, optionally wherein the ASN-B2 gene comprises the nucleotide sequence of SEQ ID NO: 57.

11. The modified wheat plant of any one of embodiments 1-10, wherein the ASN-D2 gene comprises the nucleotide sequence of SEQ ID NO: 46, 48, 49, or 51, optionally wherein the ASN-D2 gene comprises the nucleotide sequence of SEQ ID NO: 53, 55, 56, or 58.

12. The modified wheat plant of any one of embodiments 1-11, wherein the wheat plant is of the variety Bobwhite, Denali, Ripper, Snowmass 2.0, or Steamboat.

13. The modified wheat plant of any one of embodiments 1-12, a sample of seed of the plant having been deposited under NCMA Accession No. ______, ______, or ______, or ______.

14. A plant part, plant cell, or seed of the modified plant of any one of embodiments 1-13.

15. A method of reducing free asparagine concentration in the grain of a wheat plant, the method comprising: introducing a loss-of-function mutation in at least one endogenous ASN2 gene selected from ASN-A2, ASN-B2, and ASN-D2.

16. The method of embodiment 15, wherein the mutation is introduced in the ASN-A2 gene and the ASN-B2 gene; the ASN-A2 gene and the ASN-D2 gene; or the ASN-B2 gene and the ASN-D2 gene.

17. The method of embodiment 15 or embodiment 16, wherein the mutation is introduced in the ASN-A2 gene, the ASN-B2 gene, and the ASN-D2 gene.

18. The method of any one of embodiments 15-17, wherein the mutation is introduced by genome editing.

19. The method of any one of embodiments 15-18, wherein the method comprises introducing a Cas9 nuclease and a guide RNA targeting the ASN2 gene.

20. The method of any one of embodiments 15-19, wherein the guide RNA targets a region in the first exon encoding the glutamine amidotransferase (GATase) domain of the ASN2 gene.

21. The method of any one of embodiments 15-20, wherein the guide RNA comprises the nucleotide sequence of SEQ ID NO: 30.

22. The method of any one of embodiments 15-21, wherein the wheat plant is of the variety Bobwhite, Denali, Ripper, Snowmass 2.0, or Steamboat.

23. A method of producing a wheat plant having grain with reduced free asparagine concentration, the method comprising: (a) crossing the plant of any one of embodiments 1-13 with itself or another plant to produce seed; and (b) growing a progeny plant from the seed to produce a plant having grain with reduced free asparagine concentration.

24. The method of embodiment 23 further comprising: (c) crossing the progeny plant with itself or another plant; and (d) repeating steps (b) and (c) for an additional 0-7 generations to produce a plant having grain with reduced free asparagine concentration.

25. A crop comprising a plurality of the plants of any one of embodiments 1-13 planted together in an agricultural field.

26. A commodity plant product prepared from the plant of any one of embodiments 1-13, or a part thereof.

27. The commodity plant product of embodiment 26, wherein the product is grain, flour, a baked good, cereal, pasta, a beverage, livestock feed, biofuel, straw, a construction material, or starch.

28. The commodity plant product of embodiment 26 or embodiment 26, wherein the product is a low acrylamide product.

29. The commodity plant product of any one of embodiments 26-28, wherein the product comprises a nucleic acid molecule comprising the nucleotide sequence of SEQ ID NO: 45, 46, 47, 48, 49, 50, or 51.

30. The commodity plant product of any one of embodiments 26-28, wherein the product comprises a nucleic acid molecule comprising the nucleotide sequence of SEQ ID NO: 52, 53, 54, 55, 56, 57, 58, or 59.

31. A method for producing a commodity plant product, the method comprising processing the plant of any one of embodiments 1-13, or a part thereof, to obtain the product.

32. The method of embodiment 31, wherein the product is grain, flour, a baked good, cereal, pasta, a beverage, livestock feed, biofuel, straw, a construction material, or starch.

33. The method of embodiment 31 or embodiment 32, wherein the product is a low acrylamide product.

34. The method of any one of embodiments 31-33, wherein the product comprises a nucleic acid molecule comprising the nucleotide sequence of SEQ ID NO: 45, 46, 47, 48, 49, 50, or 51, optionally wherein the product comprises a nucleic acid molecule comprising the nucleotide sequence of SEQ ID NO: 52, 53, 54, 55, 56, 57, 58, or 59.

35. A primer pair having the nucleotide sequences of SEQ ID NOs: 14 and 15, SEQ ID NOs: 39 and 40, SEQ ID NOs: 41 and 42, or SEQ ID NOs: 43 and 44.

36. A method of determining the presence of a loss-of-function mutation in an ASN-A2 gene, an ASN-B2 gene, or an ASN-D2 gene in a wheat plant comprising: assaying a nucleic acid sample from the wheat plant with at least one primer pair of embodiment 35.

37. A guide RNA for editing an ASN2 gene comprising the nucleotide sequence of SEQ ID NO: 30.

38. A DNA polynucleotide encoding the guide RNA of embodiment claim 37.

39. A vector encoding the guide RNA of embodiment 37 or comprising the DNA polynucleotide of embodiment 38.

40. A wheat plant cell comprising the guide RNA of embodiment 37, the DNA polynucleotide of embodiment 38, or the vector of embodiment 39, optionally wherein the cell comprises a Cas9 nuclease.

41. A nucleic acid molecule comprising the nucleotide sequence of SEQ ID NO: 45, 46, 47, 48, 49, 50, or 51.

42. A nucleic acid molecule comprising the nucleotide sequence of SEQ ID NO: 52, 53, 54, 55, 56, 57, 58, or 59.

43. A wheat plant or plant cell comprising at least one nucleic acid molecule of embodiment 41 or embodiment 42.

44. The wheat plant or plant cell of embodiment 43, wherein the wheat plant or plant cell comprises the nucleic acid molecule of SEQ ID NO: 59.

45. The wheat plant or plant cell of embodiment 43, wherein the wheat plant or plant cell comprises the nucleic acid molecules of SEQ ID NOs: 45 and 46, optionally wherein the wheat plant or plant cell comprises the nucleic acid molecules of SEQ ID NOs: 52 and 53.

46. The wheat plant or plant cell of embodiment 43, wherein the wheat plant or plant cell comprises the nucleic acid molecules of SEQ ID NOs: 47 and 48, optionally wherein the wheat plant or plant cell comprises the nucleic acid molecules of SEQ ID NOs: 54 and 55.

47. The wheat plant or plant cell of embodiment 43, wherein the wheat plant or plant cell comprises the nucleic acid molecules of SEQ ID NOs: 47 and 49, optionally wherein the wheat plant or plant cell comprises the nucleic acid molecules of SEQ ID NOs: 54 and 56.

48. The wheat plant or plant cell of embodiment 43, wherein the wheat plant or plant cell comprises the nucleic acid molecules of SEQ ID NOs: 45, 50, and 51, optionally wherein the wheat plant or plant cell comprises the nucleic acid molecules of SEQ ID NOs: 52, 57, and 58.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1: Induced Mutations in ASPARAGINE SYNTHETASE-A2 Reduce Free Asparagine Concentration in the Wheat Grain

In this example, three backcross populations segregating for Ethyl-MethaneSulfonate (EMS)-induced null alleles of ASN-A2 were developed in durum and common wheat. In field trials, mutant lines exhibited significant reductions in free asparagine concentrations in their grain compared to wild-type sister lines but showed no significant changes in agronomic or quality traits. These non-transgenic asn-a2 null alleles can be utilized in breeding programs to help develop wheat cultivars with reduced acrylamide-forming potential.

### Characterization of Asn-a2 Null Alleles

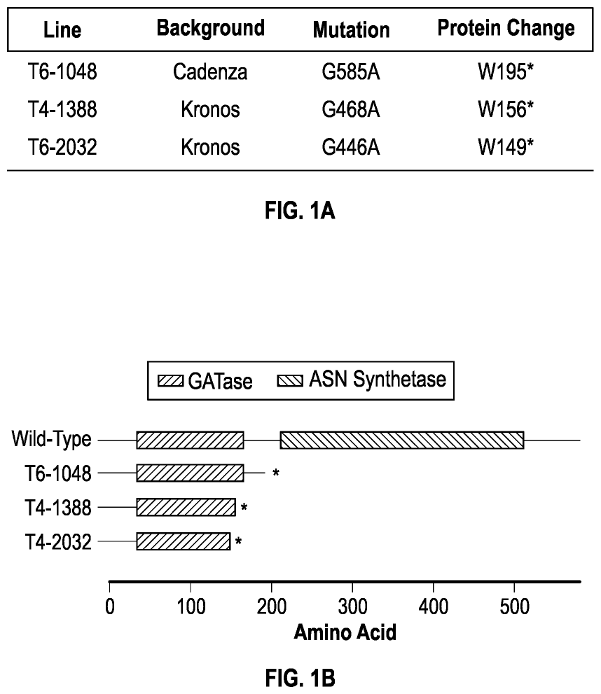

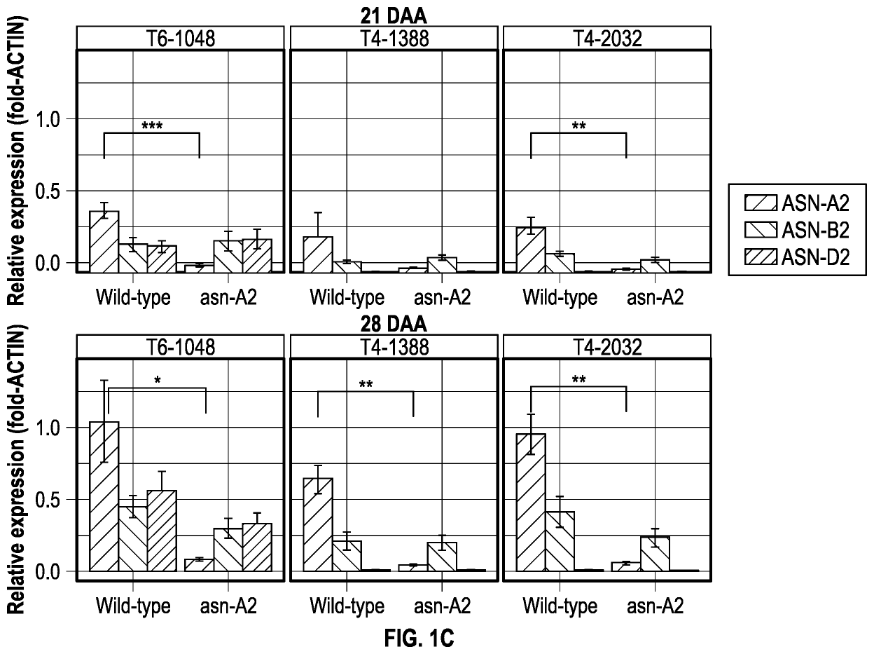

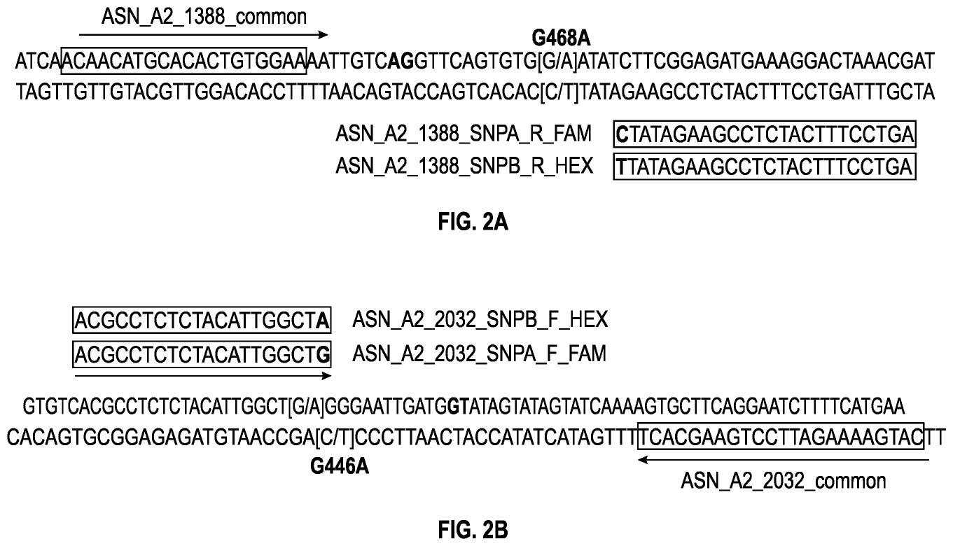

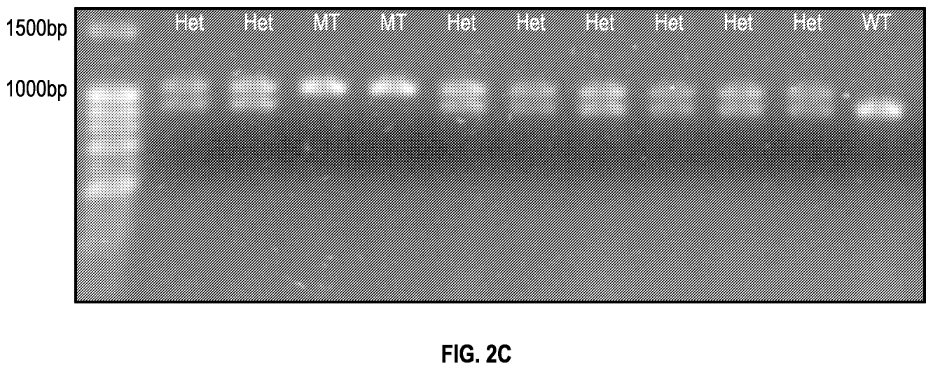

Two EMS-mutagenized lines in tetraploid Kronos (T4-1388 and T4-2032) and one in hexaploid Cadenza genetic backgrounds (T6-1048) were identified that carry point mutations predicted to introduce premature stop codons in the ASN-A2 coding region (FIG. 1A). All three lines are predicted to encode C-terminally truncated ASN-A2 proteins lacking the entire ASN-synthetase domain (FIG. 1B), so are highly likely to encode non-functional proteins. Both Kronos and Cadenza carry a complete ASN-B2 gene, so T4-1388 and T4-2032 mutant plants carry one functional ASN2 gene (TdASN-B2) and T6-1048 mutant plants carry two functional ASN2 genes (TaASN-B2 and TaASN-D) 2). Segregating BC₁F₂: 3 and BC₂F₂: 3 populations for each of these null alleles were developed by backcrossing to the corresponding wild-type parental line, selecting mutant alleles using genotyping assays. Kompetitive Allele Specific PCR (KASP) assays were developed to genotype the G468A mutation in line T4-1388 (FIG. 2A) and the G446A mutation in line T4-2032 (FIG. 2B). The G585A mutation in line T6-1048 was genotyped using a Cleaved Amplified Polymorphic Sequences (CAPS) marker (FIG. 2B). Primers for each assay are listed in TABLE 4. The full length sequence of the ASN-A2 gene of T6-1048 is provided in SEQ ID NO: 59.

In wild-type plants of all three families, ASN-A2 was the most highly expressed homeologue in grain tissues, and transcript levels rose between 21 days after anthesis (DAA) and 28 DAA (FIG. 1C). Plants carrying asn-a2 mutations exhibited significantly lower ASN-A2 transcript levels (P<0.05) than wild-type plants at 28 DAA in all three families (FIG. 1C). By contrast, ASN-B2 and ASN-1) 2 transcript levels were not significantly different (P>0.05) between wild-type and asn-a2 mutant genotypes at either timepoint (FIG. 1D).

### Effect of Asn-a2 Mutations on Free Asparagine Concentration

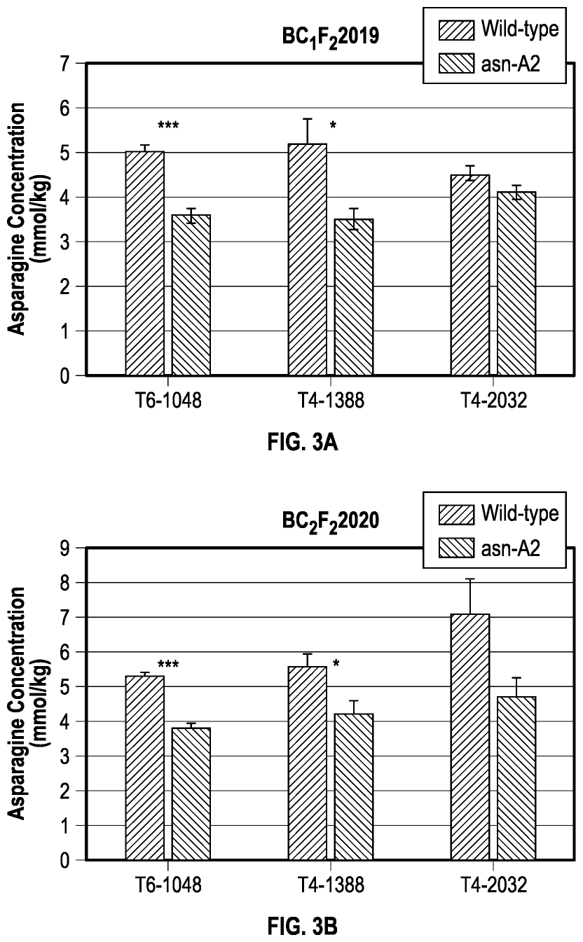

To assay the effect of the asn-a2 mutations on free asparagine concentrations, mature grain was harvested from field-grown headrows of BC₁F_{2:3} sister lines from each population as five biological replicates. Free asparagine concentration was significantly lower in asn-a2 mutants compared to wild-type sister lines in the populations derived from line T6-1048 (29% reduction, P<0.001) and line T4-1388 (33% reduction, P<0.05, FIG. 3A). In the population derived from line T4-2032, asparagine concentration was 9% lower in the asn-a2 mutant plants, but the difference was not significant (P=0.198, FIG. 3A). These results were consistent with BC₂F₂ populations grown in 2020 at the same location as headrows in six biological replicates (FIG. 3B). Free asparagine concentration was significantly lower in asn-a2 mutants compared to wild-type sister lines in the populations derived from line T6-1048 (28% reduction, P<0.0001) and line T4-1388 (24% reduction, P<0.05). In line T4-2032, although the reduction in asparagine concentration in asn-a2 mutant plants was proportionally greater than in the other populations (34% reduction) these differences were not significant due to higher variation (P=0.071, FIG. 3B). Across both years of the experiment, genotype was highly significantly associated with free asparagine concentration (P<0.0001).

### Effect of Asn-a2 Mutations on Agronomic Traits

To determine the impact of the asn-a2 mutations on plant growth and development, agronomic traits were analyzed in grains harvested from field-grown BC₂F_{2:3} plants. TABLE 2 shows the effect of asn-a2 mutations on agronomic traits in families derived from three mutant lines. Data represents the mean±standard error for six biological replicates, except for kernel diameter in the family derived from mutant line T4-2032, where five biological replicates were used. Genotype effect represents significance of wild-type vs. asn-a2 genotypes across all lines for each trait.

Germination rates were above 84% in all samples and there were no significant differences between wild-type and asn-a2 genotypes, measured 5, 7 and 14 days after sowing. In line T6-1048, the germination rate was between 2.7% and 3% lower in asn-a2 mutants compared to wild-type sister lines, although the differences were not significant. Conversely, germination rate was slightly higher in the asn-a2 mutant compared to wild-type sister lines in T4-1388 and T4-2032 populations. Despite variation in spikelet number, grain weight and diameter between Cadenza and Kronos genotypes, there were no significant differences in these traits between wild-type and asn-a2 mutant sister plants in any of the three lines.

### Effect of Asn-a2 Mutations on Quality and Breadmaking Traits

The impact of the asn-a2 null alleles on quality traits were tested using the Single Kernel Characterization System (SKCS) and NIR protein analysis, which revealed there were no significant differences between wild-type and asn-a2 sister lines for grain moisture, hardness, grain protein concentration, whole grain meal protein concentration or whole grain meal ash concentration. Solvent Retention Capacity (SRC) analysis revealed no significant differences between wild-type and mutant genotypes in lactic acid, carbonate, water or sucrose, indicating these mutations have no impact on pentosan or gliadin content in the grains, or on starch and gluten quality.

A mixograph was run on each sample to measure dough rheological properties and showed that asn-a2 and wild-type sister lines exhibited no significant differences for peak mixing time, a measure of dough strength. Peak value % was lower in asn-a2 mutants than wild-type sister plants in all three lines, although this difference was significant only in line T4-2032. Right slope (%/min) values were inconsistent between lines and were slightly higher (less negative) in asn-a2 mutants in lines T6-1048 and T4-1388, but significantly lower (more negative) in line T4-2032. Right-width % was not significantly different between genotypes in any line, indicating that the asn-a2 mutations do not confer differences in mixing tolerance.

TABLE 3 shows the effect of asn-a2 mutations on quality traits in families derived from the three mutant lines. Data represent the mean±standard error for six biological replicates. Significant effects are highlighted in bold. Genotype effect represents significance of wild-type vs. asn-a2 genotypes across all lines for each trait. SRC=Solvent Retention Capacity.

TABLE 2

| | | | | | | | | | | Genotype |
|---|---|---|---|---|---|---|---|---|---|---|
| | T6-1048 | | | T4-1388 | | | T4-2032 | | | effect |
| Trait | Wild-type | asn-a2 | P | Wild-type | asn-a2 | P | Wild-type | asn-a2 | P | P |
| Germination | 89.0 ± 1.6 | 86.3 ± 1.2 | 0.214 | 88.3 ± 1.7 | 89.3 ± 1.0 | 0.629 | 84.7 ± 1.6 | 87.0 ± 1.6 | 0.329 | 0.862 |
| 5 d (%) | | | | | | | | | | |
| Germination | 92.0 ± 1.8 | 89.0 ± 1.2 | 0.198 | 91.0 ± 1.7 | 94.0 ± 1.3 | 0.186 | 86.0 ± 1.8 | 91.0 ± 1.7 | 0.070 | 0.264 |
| 7 d (%) | | | | | | | | | | |
| Germination | 92.7 ± 1.6 | 89.7 ± 1.1 | 0.153 | 92.7 ± 1.6 | 94.7 ± 0.7 | 0.277 | 86.3 ± 1.9 | 91.7 ± 1.5 | 0.052 | 0.315 |
| 14 d (%) | | | | | | | | | | |
| Spikelet | 20.6 ± 0.1 | 20.2 ± 0.1 | 0.131 | 15.1 ± 0.2 | 14.9 ± 0.2 | 0.660 | 14.9 ± 0.3 | 14.9 ± 0.1 | 1 | 0.301 |
| number | | | | | | | | | | |
| Kernel | 24.9 ± 1.2 | 26.4 ± 0.7 | 0.324 | 33.2 ± 0.8 | 32.8 ± 1.2 | 0.772 | 33.8 ± 0.8 | 33.8 ± 0.9 | 0.992 | 0.667 |
| weight (mg) | | | | | | | | | | |
| Kernel | 2.50 ± 0.05 | 2.5 ± 0.4 | 0.439 | 2.8 ± 0.04 | 2.8 ± 0.06 | 0.956 | 2.8 ± 0.04 | 2.83 ± 0.05 | 0.870 | 0.727 |
| diameter (mm) | | | | | | | | | | |

TABLE 3

| | | | | | | | | | | Genotype |
|---|---|---|---|---|---|---|---|---|---|---|
| | T6-1048 | | | T4-1388 | | | T4-2032 | | | effect |
| Trait | Wild-type | asn-a2 | P | Wild-type | asn-a2 | P | Wild-type | asn-a2 | P | P |
| Hardness index | 74.4 ± 0.8 | 73.2 ± 1.0 | 0.373 | 79.1 ± 1.0 | 77.2 ± 1.1 | 0.239 | 79.4 ± 1.2 | 77.9 ± 1.7 | 0.488 | 0.110 |
| Grain moisture % | 6.28 ± 0.06 | 6.17 ± 0.06 | 0.231 | 6.16 ± 0.06 | 6.11 ± 0.06 | 0.547 | 6.03 ± 0.04 | 6.08 ± 0.04 | 0.472 | 0.421 |
| Whole grain meal | 2.51 ± 0.08 | 2.43 ± 0.05 | 0.410 | 2.14 ± 0.03 | 2.14 ± 0.03 | 0.969 | 2.03 ± 0.02 | 2.09 ± 0.05 | 0.286 | 0.864 |
| ash (14% MB) | | | | | | | | | | |
| Whole grain meal | 18.3 ± 0.2 | 17.8 ± 0.2 | 0.163 | 18.0 ± 0.3 | 17.9 ± 0.3 | 0.666 | 18.3 ± 0.2 | 18.6 ± 0.3 | 0.457 | 0.597 |
| protein (14% MB) | | | | | | | | | | |
| Grain Protein % | 18.7 ± 0.2 | 18.3 ± 0.2 | 0.234 | 18.1 ± 0.3 | 18.1 ± 0.3 | 0.946 | 18.9 ± 0.3 | 19.1 ± 0.3 | 0.686 | 0.778 |
| (12% MB) | | | | | | | | | | |
| Water SRC % | 77.8 ± 1.6 | 77.0 ± 1.4 | 0.705 | 83.4 ± 0.8 | 84.5 ± 1.2 | 0.488 | 85.0 ± 0.6 | 85.4 ± 0.8 | 0.705 | 0.818 |
| Lactic acid SRC % | 101.3 ± 1.9 | 100.3 ± 2.1 | 0.741 | 113.2 ± 1.8 | 112.2 ± 2.5 | 0.752 | 116.6 ± 1.8 | 115.0 ± 2.2 | 0.589 | 0.486 |
| Sucrose SRC % | 121.4 ± 1.5 | 118.7 ± 1.2 | 0.195 | 130.9 ± 1.2 | 130.7 ± 1.4 | 0.898 | 133.8 ± 1.8 | 134.1 ± 1.4 | 0.880 | 0.466 |
| Carbonate SRC % | 105.8 ± 1.3 | 104.3 ± 0.9 | 0.343 | 107.6 ± 2.0 | 107.8 ± 1.0 | 0.908 | 110.8 ± 1.6 | 108.7 ± 1.3 | 0.348 | 0.338 |
| Peak time (min) | 3.59 ± 0.12 | 3.34 ± 0.08 | 0.099 | 3.64 ± 0.13 | 3.74 ± 0.16 | 0.661 | 3.91 ± 0.16 | 3.56 ± 0.09 | 0.088 | 0.103 |
| Peak value % | 47.3 ± 0.9 | 46.7 ± 0.4 | 0.509 | 49.5 ± 0.5 | 48.7 ± 0.4 | 0.280 | 48.4 ± 0.3 | 47.1 ± 0.3 | 0.010 | 0.044 |
| Left slope (%/min) | 9.3 ± 0.6 | 9.5 ± 0.7 | 0.759 | 10.0 ± 0.5 | 9.4 ± 0.6 | 0.468 | 10.2 ± 0.7 | 10.8 ± 0.5 | 0.510 | 0.877 |
| Right slope (%/min) | −1.85 ± 0.23 | −1.75 ± 0.21 | 0.752 | −3.79 ± 1.24 | −2.68 ± 0.33 | 0.408 | −1.99 ± 0.22 | −3.08 ± 0.13 | 0.003 | 0.931 |
| Right width % | 11.9 ± 1.4 | 11.2 ± 0.7 | 0.652 | 26.0 ± 2.6 | 24.2 ± 1.8 | 0.587 | 25.6 ± 2.2 | 24.3 ± 2.4 | 0.697 | 0.427 |

### Free Asparagine Concentration in Wheat Varieties Adapted to the Great Plains

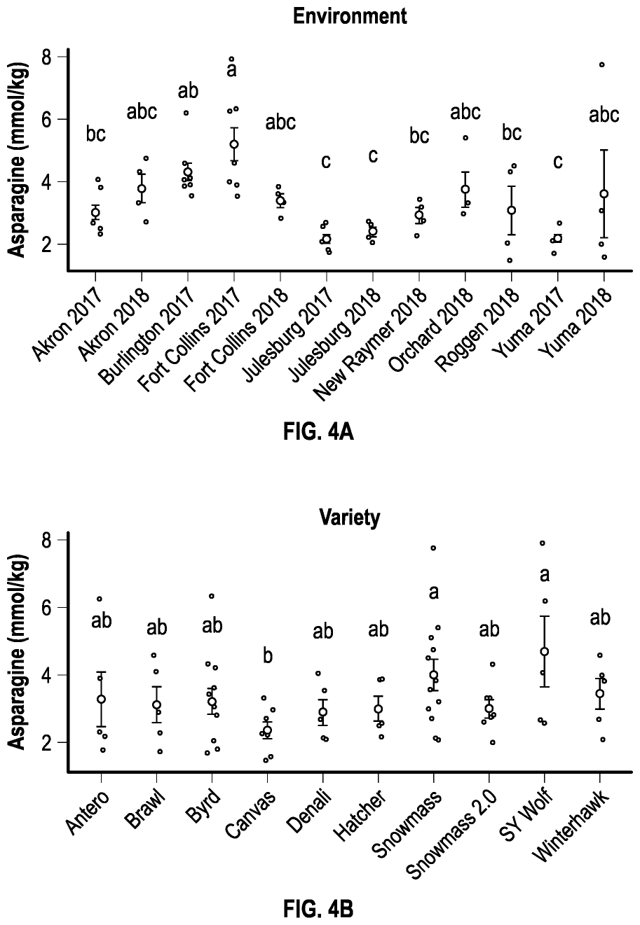

To better understand the opportunities to reduce free asparagine concentration in breeding programs, free asparagine concentration was measured in grain samples of elite winter wheat germplasm selected for their economic importance and acreage in Colorado and the Great Plains. Six winter wheat varieties were assayed in five field locations in 2017 (FIG. 4A) and four varieties were assayed in seven locations in 2018 (FIG. 4B). Asparagine concentrations varied by environment, with higher values in varieties grown in Fort Collins 2017 compared to Julesberg or Yuma, but comparatively smaller differences between these environments in 2018. Some genotypes showed consistent free asparagine concentrations between environments. In 2017, ‘SY Wolf’ consistently exhibited high asparagine concentration in different environments, whereas in 2018, Snowmass exhibited the highest free asparagine concentration of all assayed varieties. The varieties ‘Snowmass’, ‘Antero’ and ‘Hatcher’ carry the TaASN-B2 deletion, while ‘Brawl’, ‘Byrd’, ‘Denali’, ‘SY Wolf’ and ‘Winterhawk’ all carry a functional copy of this gene. In neither year was there a correlation between the presence or absence of TaASN-B2 and mean asparagine concentration, indicating that other genotypic and environmental factors are driving the observed variation in free asparagine concentration among these wheat varieties.

### Induced Asn-a2 Mutations Confer Reduced Free Asparagine Concentration

Although natural genetic variation for free asparagine concentration exists in wheat germplasm collections, this is a highly quantitative trait determined by multiple small-effect QTL, complicating the identification, characterization and deployment of natural variants. As a complementary approach, in silico databases of chemically-mutagenized wheat populations provide rapid access to novel genetic variation that can be deployed in breeding programs without regulatory oversight, for example, to develop high-amylose wheat varieties. One drawback of mutagenized populations is the confounding effect of residual background mutations, since tetraploid and hexaploid M4 plants carry, on average, 2,705 and 5,351 exonic point mutations, respectively. Even though two backcrosses were performed in the current study, the variable effect of these background mutations may account for phenotypic variation in line T4-2032 such that the differences in free asparagine concentration between wild-type and mutant sister lines were not significant (FIG. 3). Despite this variation, consistent reductions in free asparagine concentrations in three independently-derived mutant populations in both tetraploid and hexaploid wheat provide strong evidence that the observed phenotype is conferred by non-functional induced mutations in ASN-A2. Furthermore, transcript levels of ASN-B2 and ASN-D2 were not affected by asn-a2 mutations in any line (FIG. 1), suggesting the impacts are due to reduced activity of ASN-A2 itself, and not secondary effects on the activity of homeologous ASN2 genes. However, these conclusions are based on results from just two environmental replications using sibling lines derived from individual mutant plants. It will be important to validate these findings using more extensive field trials replicated in both space and time and using independently derived populations segregating for variation in TaASN2 genes. The introgression of the asn-a2 null allele into Snowmass 2.0 has been initiated to characterize its effect in an elite winter wheat variety.

The reduction in free asparagine concentration in the asn-a2 mutants ranged from 9% to 34% (FIG. 3), comparable to the 16.2% reduction associated with a natural deletion of TaASN-B2 in S-sufficient conditions, but much lower than the 90% reduction observed in one CRISPR/Cas edited plant carrying mutations in all three ASN2 homeologues (Raffan et al., Plant Biotechnol J. 2021 August; 19 (8): 1602-1613). The polyploid wheat genome provides functional redundancy, so it will be interesting to characterize isogenic materials with different combinations of ASN2 alleles to reveal the relative impacts of each homeologue and the extent to which they act additively to reduce free asparagine concentration. These alleles may also be combined with independent natural QTL elsewhere in the genome to further lower free asparagine concentration.

When integrating novel genetic variation, it is essential to determine possible pleiotropic effects on other traits. Most notably, CRISPR-edited plants with the greatest reductions in free asparagine concentration exhibited poor germination rates (Raffan et al., Plant Biotechnol J. 2021 August; 19 (8): 1602-1613), which may limit their utility for wheat breeders and growers. By contrast, the asn-a2 mutant lines described in this example exhibit no significant reduction in germination rate (TABLE 2), suggesting that the asparagine concentration in these seeds is sufficient for normal germination. A comprehensive quality analysis including SRC, whole grain meal and mixograph analyses revealed that asn-a2 mutant lines exhibit no detrimental effects for quality traits compared to wild-type plants (TABLE 3). These findings are consistent with previous analyses of the impacts of natural variation in free asparagine concentration on these traits, where only mild negative correlations between sedimentation volume (a proxy for the quality traits measured here) and free asparagine concentration. These results are encouraging and support the hypothesis that it should be possible to breed for mild reductions in asparagine concentration without compromising on baking quality or yield.

### Reducing Free Asparagine Concentration in Other Species

These findings show the potential to apply induced or natural variation in ASN genes to reduce asparagine concentrations in other species. Characterizing the expression profiles and natural variation of ASN2 orthologs in barley and rye could reveal opportunities to reduce free asparagine concentration in the grain of these species. However, because of their diploid genomes, the strategy to induce more subtle reductions in asparagine concentration by targeting different combinations of homeologous genes would not be possible, and instead, milder variants that reduce, but do not abolish, gene activity may be more valuable.

The duplication event that gave rise to TaASN1 and TaASN2 genes occurred only in the Triticeae lineage, meaning that the genomes of other monocots, including rice, maize and sorghum, have no ASN2 ortholog. The rice genome contains two asparagine synthetase genes, so induced variation in either of these genes is likely to confer major changes in plant fitness not observed in wheat due to functional redundancy. However, in potato, while suppressing expression of both StASN1 and StASN2 had detrimental effects on growth and development, a more targeted approach focused only on StASN1 reduced tuber asparagine content without other undesirable phenotypes, showing that it would be worthwhile exploring genetic diversity in the ASN gene family in other species.

### Characterizing the ASN Gene Family in Wheat

Within wheat, reverse genetics approaches using either TILLING or CRISPR/Cas can also be applied to characterize other members of the TaASN gene family. In addition to their functional characterization, this approach may reveal novel combinations of alleles that could be beneficial for acrylamide reduction in wheat. Of note are TaASN3.1 genes that are expressed during early embryo, ovule and grain development. Searches of the in silico TILLING database revealed that there are multiple Kronos and Cadenza lines carrying point mutations encoding premature stop codons for all three homoeologous copies of TaASN-3.1. These mutant materials could be developed to test the hypothesis that ASN-3.1 contributes to free asparagine concentrations in the wheat grain. Combining these alleles with ASN2 mutations could deliver further reductions in free asparagine concentrations in the grain. However, TaASN3.1 genes are also expressed in leaf and stem tissues, so knockout alleles may have pleotropic effects on plant health and development.

TABLE 4

| | Amplified | | Primer Sequence | Amplicon |
|---|---|---|---|---|
| Purpose | region | Primer Name | (5′-3′) | size (bp) |
| Check for the | Region | ASN-B2_CS_F3 | AGCAAGCCTTCACCATCATT | 189 |
| absence of | flanking | | (SEQ ID NO: 10) | |
| ASN-B2 | deleted | ASN-B2_CS_R1 | GATGTAGGCATGTCAACGAGA | |
| | region in | | (SEQ ID NO: 11) | |
| | varieties | | | |
| | with no | | | |
| | ASN-B2 | | | |
| Check for the | Exon 11-3′ | ASN-B2_qF1 | AACAAGCCTGGGGTGATGAG | 125 |
| presence of | UTR | | (SEQ ID NO: 12) | |
| ASN-B2 | | ASN-B2_qR1 | TTGTCTCAAAAAGAAAAAGAA | |
| | | | CTTG (SEQ ID NO: 13) | |
| T6-1048 | Region | ASN-A2_1048_F3 | AAGAAGAAAGTGACCACACA | 1,063 |
| CAPS marker | from | | (SEQ ID NO: 14) | |
| | intron 1 to | ASN-A2_1048_R3 | TTTTCAAAGATACTAGCACATA | |
| | 3 | | AT (SEQ ID NO: 15) | |
| T4-1388 | Region | ASN_A2_1388_ | GAAGGTGACCAAGTTCATGCTA | 62 |
| KASP marker | from intron | SNPA_R_FAM | GTCCTTTCATCTCCGAAGATAT | |
| | 2 to exon 3 | | C (SEQ ID NO: 16) | |
| | | ASN_A2_1388_ | GAAGGTCGGAGTCAACGGATT | |
| | | SNPB_R_HEX | AGTCCTTTCATCTCCGAAGATA | |
| | | | TT (SEQ ID NO: 17) | |
| | | ASN_A2_1388 | ACAACATGCACACTGTGGAA | |
| | | _common | (SEQ ID NO: 18) | |
| T4-2032 | Region | ASN_A2_2032_ | GAAGGTGACCAAGTTCATGCTA | 66 |
| KASP marker | from exon | SNPA_F_FAM | CGCCTCTCTACATTGGCTG | |
| | 2 to intron | | (SEQ ID NO: 19) | |
| | 2 | ASN_A2_2032_ | GAAGGTCGGAGTCAACGGATT | |
| | | SNPB_F_HEX | ACGCCTCTCTACATTGGCTA | |
| | | | (SEQ ID NO: 20) | |
| | | ASN_A2_2032_ | CATGAAAAGATTCCTGAAGCAC | |
| | | common | T (SEQ ID NO: 21) | |
| ASN-A2 | Exons 1 and | ASN-A2_F | TCAACGGGGAGGTCTACAAC | 184 |
| expression | 2 | | (SEQ ID NO: 22) | |
| | | ASN-A2_R | GCAATGAAGCTGTTATCTCGTG | |
| | | | (SEQ ID NO: 23) | |
| ASN-B2 | Exons 1 and | ASN-B2_F | GTCAACGGGGAGATCTACAACC | 185 |
| expression | 2 | | (SEQ ID NO: 24) | |
| | | ASN-B2_R | GCGATGAAGCTGTGATCTCTTG | |
| | | | (SEQ ID NO: 25) | |
| ASN-D2 | Exons 1 and | ASN-D2_F | GTGAACGGGGAGATTTACAAC | 185 |
| expression | 2 | | (SEQ ID NO: 26) | |
| | | ASN-D2_R | GCAATGAAGCTCTTATCTCGTG | |
| | | | (SEQ ID NO: 27) | |
| Housekeeping | | ACTIN-F | CAGAGTCGAGCACAATACCAGT | 91 |
| gene | | | TG (SEQ ID NO: 28) | |
| | | ACTIN-R | ACCTTCAGTTGCCCAGCAAT | |
| | | | (SEQ ID NO: 29) | |

### Example 2: Asparagine Synthetase 2 (ASN2) Editing in Wheat to Reduce Asparagine Concentration in Wheat Grain

To develop wheat varieties with reduced acrylamide-forming potential, a CRISPR/Cas9 construct was developed to induce non-functional variants in TaASN2 genes. The wheat genome contains 15 TaASN genes that encode ASPARAGINE SYNTHETASE enzymes required for asparagine biosynthesis. The three TaASN2 homologues are notable for their grain-specific expression profile.

### CRISPR/Cas9 Construct Design and Assembly

A CRISPR/Cas9 construct was designed containing a single guide RNA (sgRNA) to generate null alleles for all three wheat TaASN2 genes (TaASN-A2, TaASN-B2 and TaASN-D2). Some varieties contain a natural deletion of TaASN-B2, but all varieties studied so far have a functional copy of TaASN-A2 and TaASN-D2. A 20 nucleotide sgRNA target (5′-GTAGAGCGGCTGGTCGCCGG-3′; SEQ ID NO: 30) was designed on the antisense strand between positions 172 and 192 bp downstream of the initiating ATG codon of ASN-A2. The sgRNA targets a region in the first exon encoding the GATase domain and upstream of the ASN synthase domain. Both these domains are critical for ASN protein function.

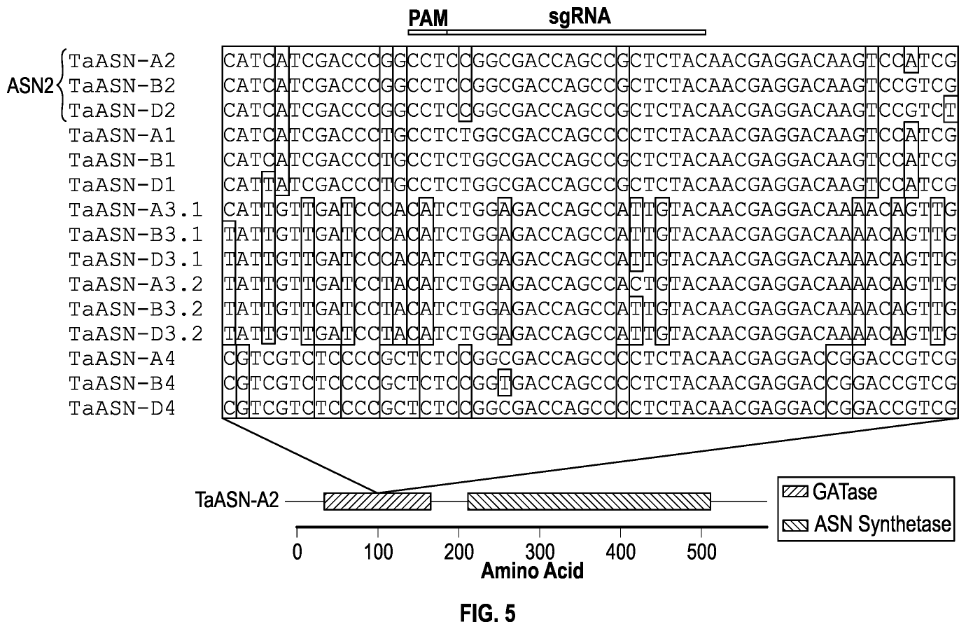

The target sequence is proceeded by an AGG Protospacer Adjacent Motif (PAM) that is essential for CRISPR/Cas9 activity (FIG. 5). The 23 nucleotide sgRNA and PAM sequence matches TaASN-A2, TaASN-B2 and TaASN-D2 genes with 100% identity. For nine of the other ASN genes, the PAM sequence was absent, meaning the construct will not edit these genes (TABLE 5). The PAM sequence was present in three ASN1 homologues, but the sgRNA contained at least one mismatch for each sequence, reducing the efficiency by which these alleles may be edited.

Furthermore, very few off-target effects are predicted. Two possible alternative targets in the protein-coding region of two genes encoding calcium Uniporter proteins (TraesCS4B02G236600 and TraesCS4A02G060400LC) were identified. However, both these genes have four mismatches across the 20-nucleotide target sequence minimizing the chance of their being edited.

TABLE 5

| | IWGSC RefSeq v2.0 | | PAM | Number of |
|---|---|---|---|---|
| Gene name | gene ID | sgRNA sequence | present? | mismatches |
| TaASN-A1 | TraesCS5A02G153900 | CCTCTGGCGACCAGCCCCTCTAC | Yes | 2 |
| | | (SEQ ID NO: 31) | | |
| TaASN-B1 | TraesCS5B02G152600 | CCTCTGGCGACCAGCCGCTCTAC | Yes | 1 |
| | | (SEQ ID NO: 32) | | |
| TaASN-D1 | TraesCS5D02G159100 | CCTCTGGCGACCAGCCGCTCTAC | Yes | 1 |
| | | (SEQ ID NO: 32) | | |
| TaASN-A2 | TraesCS3A02G077100 | CCTCCGGCGACCAGCCGCTCTAC | Yes | |
| | | (SEQ ID NO: 33) | | |
| TaASN-B2 | Not present | CCTCCGGCGACCAGCCGCTCTAC | Yes | |
| | | (SEQ ID NO: 33) | | |
| TaASN-D2 | TraesCS3D02G077300 | CCTCCGGCGACCAGCCGCTCTAC | Yes | |
| | | (SEQ ID NO: 33) | | |
| TaASN-A3.1 | TraesCS1A02G382800 | CATCTGGAGACCAGCCATTGTAC | No | 5 |
| | | (SEQ ID NO: 34) | | |
| TaASN-B3.1 | TraesCS1B02G408200 | CATCTGGAGACCAGCCATTGTAC | No | 5 |
| | | (SEQ ID NO: 34) | | |
| TaASN-D3.1 | TraesCS1D02G390500 | CATCTGGAGACCAGCCATTGTAC | No | 5 |
| | | (SEQ ID NO: 34) | | |
| TaASN-A3.2 | TraesCS1A02G422100 | CATCTGGAGACCAGCCACTGTAC | No | 5 |
| | | (SEQ ID NO: 35) | | |
| TaASN-B3.2 | TraesCS1B02G453600 | CATCTGGAGACCAGCCATTGTAC | No | 5 |
| | | (SEQ ID NO: 34) | | |
| TaASN-D3.2 | TraesCS1D02G430300 | CATCTGGAGACCAGCCATTGTAC | No | 5 |
| | | (SEQ ID NO: 34) | | |
| TaASN-A4 | TraesCS4A02G109900 | TCTCCGGCGACCAGCCCCTCTAC | No | 3 |
| | | (SEQ ID NO: 36) | | |
| TaASN-B4 | TraesCS4B02G194400 | TCTCCGGTGACCAGCCCCTCTAC | No | 4 |
| | | (SEQ ID NO: 37) | | |
| TaASN-D4 | TraesCS4D02G195100 | TCTCCGGTGACCAGCCCCTCTAC | No | 4 |
| | | (SEQ ID NO: 37) | | |

### CRISPR/Cas9 Plasmid Assembly and Transformation

The 20 nucleotide sgRNA sequence was synthesized as overlapping, complementary oligos with overhanging 5′ and 3′ ends complementary to the insertion site of the target vector. Oligos were hybridized and cloned into the JD633 vector by Golden Gate cloning following vector AarI digestion. This sgRNA was integrated immediately downstream of the U6 promoter, and the vector also contains ZmUbi1::SpCas9 and TaGRF4:TaGIF1 coding sequences which confer improved regeneration rates in transformed callus tissue (Debernardi et al., Nat Biotechnol. 2020 November; 38 (11): 1274-1279). Ligated vectors were confirmed by Sanger sequencing and transformed into DH5-a E. coli cells from which purified plasmid DNA was extracted. After confirming sequence insertion and integrity by Sanger sequencing, plasmid DNA was transformed into Agrobacterium tumefaciens strain AGL1 by electroporation and transformed into seven wheat genotypes; Bobwhite, Denali, Ripper, Snowmass 2.0, Steamboat, CO16D402W, and CO18D181R following an embryo transformation protocol. Transgenic wheat plants were selected on hygromycin media and following regeneration were validated by PCR assays to amplify two fragments of the transformed plasmid (a region of the U6 promoter, and a fragment of the hpt gene).

### Characterizing Editing Events

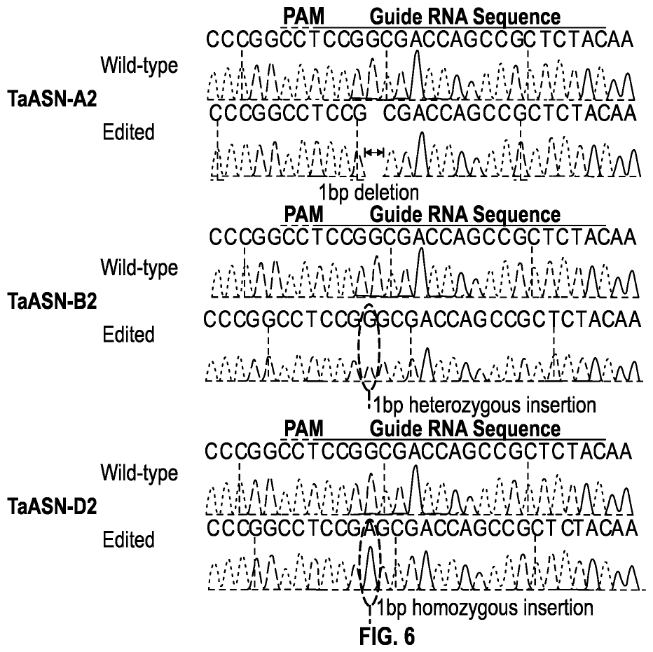

Homologue-specific PCR primers were designed to amplify and sequence each of the three target TaASN genes to characterize induced edits (TABLE 6). In CO18D181R, this construct induced disruptive edits in all three TaASN2 genes (FIG. 6). All edits were found in the characteristic position 3-4 nucleotides upstream of the PAM. The construct induced a homozygous one-nucleotide deletion in TaASN-A2, and a homozygous one-nucleotide insertion in TaASN-D2. The induced edits in TaASN-B2 were heterozygous. To interpret these edited alleles, the PCR amplicon was cloned into the pGEM-T vector and transformed into E. coli bacterial cells followed by sequencing individual colonies containing single PCR fragments. One TaASN-B2 edited allele contains a one-nucleotide insertion, while the other allele contains a one nucleotide deletion. All induced edits disrupt the open reading frame of the gene's protein coding region, and should disrupt translation of the critical ASN synthetase domain, so are expected to encode non-functional alleles.

TABLE 6

| Target gene | Forward primer (5′-3′) | Reverse primer (5′-3′) |
|---|---|---|
| TaASN-A2 | CATGGACGACGTGTAGCCCT | TTGTATGCTAGAAGCTACGGATTGAC |
| | (SEQ ID NO: 39) | (SEQ ID NO: 40) |
| TaASN-B2 | GAGAGGAAAGCAGAGCAGTCTT | ACCTTCCGACATGTTTCCTG |
| | (SEQ ID NO: 41) | (SEQ ID NO: 42) |
| TaASN-D2 | TCCAGAGGAGCAGCATAACC | AAGAATGGTGGTGTGTGC |
| | (SEQ ID NO: 43) | (SEQ ID NO: 44) |

The CRISPR/Cas9 construct also edited ASN2 genes with a high rate of efficiency in six other wheat varieties; Bobwhite, Denali, Ripper, Snowmass 2.0, Steamboat and CO16D402W. TABLE 7 provides a summary of the edited ASN2 genes in these varieties. The sgRNA-PAM sequence shares 100% identity in all ASN2 genes present in fifteen wheat varieties with assembled genomes, suggesting this construct can be applied broadly in diverse germplasm, including those containing a functional copy of ASN-B2 and those without.

TABLE 7

| | | Edited ASN-A2 | | Edited ASN-B2 | | Edited ASN-D2 | |
|---|---|---|---|---|---|---|---|
| | | | Full | | Full | | Full |
| | | Exon 1 | Length | Exon 1 | Length | Exon 1 | Length |
| Genotype ID | Variety | Sequence | Sequence | Sequence | Sequence | Sequence | Sequence |
| AK125D.1 | Bobwhite | SEQ ID | SEQ ID | Deleted | | SEQ ID | SEQ ID |
| | | NO: 45 | NO: 52 | | | NO: 46 | NO: 53 |
| AK167.1.2 | Snowmass | SEQ ID | SEQ ID | Deleted | | SEQ ID | SEQ ID |
| | 2.0 | NO: 47 | NO: 54 | | | NO: 48 | NO: 55 |
| AK160.2.6 | Steamboat | SEQ ID | SEQ ID | Deleted | | SEQ ID | SEQ ID |
| | | NO: 47 | NO: 54 | | | NO: 48 | NO: 55 |
| AK108.2.1.4 | Ripper | SEQ ID | SEQ ID | Unedited | | SEQ ID | SEQ ID |
| | | NO: 47 | NO: 54 | | | NO: 49 | NO: 56 |
| AK109b.4.2.2 | Denali | SEQ ID | SEQ ID | SEQ ID | SEQ ID | SEQ ID | SEQ ID |
| | | NO: 45 | NO: 52 | NO: 50 | NO: 57 | NO: 51 | NO: 58 |

### Phenotypic Characterization

To characterize the impact of these induced mutations on free asparagine concentration, crosses between edited lines homozygous for null alleles at the ASN2 loci and the corresponding wild-type parental line have been initiated. From these progeny, individuals carrying edited ASN2 alleles but lacking any transgenic insertion will be selected. Lines with different combinations of ASN2 alleles will be selected and grown as headrows in replicated field experiments to quantify free asparagine concentration, agronomic and quality traits of mutant lines compared to wild-type sister lines of the same genotype.

### Applications in Breeding

Experimentally, the CRISPR/Cas9 construct induces frame-shift mutations in all ASN2 genes in seven wheat varieties. Analysis of assembled wheat genomes shows that the sgRNA-PAM site is conserved in all ASN2 genes found in the genomes of 15 wheat varieties sequenced so far, indicating that the CRISPR construct can likely be applied broadly across wheat varieties, including those that carry a functional copy of the ASN-B2 gene. There is a limited risk of off-target mutations elsewhere in the wheat genome. Editing these genes to generate lines with reduced free asparagine concentration would accelerate the development of varieties with low acrylamide forming potential, which is complicated to achieve using natural variation due to the limited natural genetic variation for this trait. The varieties developed may be marketed at a premium to be processed into healthier, low acrylamide bread products.

### DEPOSIT

Applicant will make a deposit of at least 625 seeds of wheat lines AK167.1.2, AK160.2.6, AK108.2.1.4, and AK109b.4.2.2 with the Provasoli-Guillard National Center for Marine Algae and Microbiota (NCMA), 60 Bigelow Drive, East Boothbay, Me. 04544, USA, with NCMA Accession No. ______, ______, ______, and ______, respectively. The seeds deposited with the NCMA on ______ will be taken from the deposit maintained by Colorado State University, Department of Soil and Crop Sciences, 307 University Ave., Fort Collins, CO 80523-1170 since prior to the filing date of this application. Access to this deposit will be available during the pendency of the application to the Commissioner of Patents and Trademarks and persons determined by the Commissioner to be entitled thereto upon request. Upon issue of claims, the Applicant(s) will make available to the public, pursuant to 37 CFR 1.808, a deposit of at least 625 seeds of wheat lines AK167.1.2, AK160.2.6, AK108.2.1.4, and AK109b.4.2.2 with the NCMA. This deposit will be maintained in the depository, which is a public depository, for a period of 30 years, or 5 years after the most recent request, or for the enforceable life of the patent, whichever is longer, and will be replaced if it becomes nonviable during that period. Additionally, Applicant will satisfy all the requirements of 37 C.F.R. §§ 1.801-1.809, including providing an indication of the viability of the sample. Applicant has no authority to waive any restrictions imposed by law on the transfer of biological material or its transportation in commerce. Applicant does not waive any infringement of their rights granted under this patent or under the Plant Variety Protection Act (7 USC 2321 et seq.).

What is claimed is:

1. A modified wheat plant, or a progeny thereof, comprising a loss-of-function mutation in at least one endogenous ASPARAGINE SYNTHETASE 2 (ASN2) gene selected from ASN-A2, ASN-B2, and ASN-D2, wherein the mutation confers reduced free asparagine concentration in the grain of the wheat plant.

2. The modified wheat plant of claim 1, wherein the plant comprises a loss-of-function mutation in the ASN-A2 gene and the ASN-B2 gene; the ASN-A2 gene and the ASN-D2 gene; or the ASN-B2 gene and the ASN-D2 gene.

3. The modified wheat plant of claim 1, wherein the plant comprises a loss-of-function mutation in the ASN-A2 gene, the ASN-B2 gene, and the ASN-D2 gene.

4. The modified wheat plant of claim 1, wherein the mutation introduces a frameshift mutation or a pre-mature stop codon in the ASN2 gene.

5. The modified wheat plant of claim 1, wherein the mutation is in the first exon encoding the glutamine amidotransferase (GATase) domain of the ASN2 gene.

6. The modified wheat plant of claim 1, wherein the mutation is an insertion, a deletion, or a substitution of one or more nucleotides from position 172 to 192 with reference to SEQ ID NO: 1, 2, or 3.

7. The modified wheat plant of claim 1, wherein the free asparagine concentration of the grain is reduced by at least 30% relative to the free asparagine concentration of grain without the mutation.

8. The modified wheat plant of claim 1, wherein the grain has a germination rate of about 70% to about 100% relative to the germination rate of grain without the mutation.

9. The modified wheat plant of claim 1, wherein the ASN-A2 gene comprises the nucleotide sequence of SEQ ID NO: 52, 54, or 59.

10. The modified wheat plant of claim 1, wherein the ASN-B2 gene comprises the nucleotide sequence of SEQ ID NO: 57.

11. The modified wheat plant of claim 1, wherein the ASN-D2 gene comprises the nucleotide sequence of SEQ ID NO: 53, 55, 56, or 58.

12. The modified wheat plant of claim 1, wherein the wheat plant is of the variety Bobwhite, Denali, Ripper, Snowmass 2.0, or Steamboat.

13. A plant part, plant cell, or seed of the modified wheat plant of any one of claims 1-12.

14. A method of reducing free asparagine concentration in the grain of a wheat plant, the method comprising:
introducing a loss-of-function mutation in at least one endogenous ASN2 gene selected from ASN-A2, ASN-B2, and ASN-D2.

15. The method of claim 14, wherein the mutation is introduced in the ASN-A2 gene and the ASN-B2 gene; the ASN-A2 gene and the ASN-D2 gene; or the ASN-B2 gene and the ASN-D2 gene.

16. The method of claim 14, wherein the mutation is introduced in the ASN-A2 gene, the ASN-B2 gene, and the ASN-D2 gene.

17. The method of claim 14, wherein the mutation is introduced by genome editing.

18. The method of claim 14, wherein the method comprises introducing a Cas9 nuclease and a guide RNA targeting the ASN2 gene.

19. The method of claim 18, wherein the guide RNA targets a region in the first exon encoding the GATase domain of the ASN2 gene.

20. The method of claim 18, wherein the guide RNA comprises the nucleotide sequence of SEQ ID NO: 30.

21. The method of claim 14, wherein the wheat plant is of the variety Bobwhite, Denali, Ripper, Snowmass 2.0, or Steamboat.

22. A method of producing a wheat plant having grain with reduced free asparagine concentration, the method comprising:
(a) crossing the plant of any one of claims 1-12 with itself or another plant to produce seed; and
(b) growing a progeny plant from the seed to produce a plant having grain with reduced free asparagine concentration.

23. The method of claim 22 further comprising:
(c) crossing the progeny plant with itself or another plant; and
(d) repeating steps (b) and (c) for an additional 0-7 generations to produce a plant having grain with reduced free asparagine concentration.

24. A crop comprising a plurality of the plants of any one of claims 1-12 planted together in an agricultural field.

25. A commodity plant product prepared from the plant of any one of claims 1-12, or a part thereof.

26. The commodity plant product of claim 25, wherein the product is grain, flour, a baked good, cereal, pasta, a beverage, livestock feed, biofuel, straw, a construction material, or starch.

27. The commodity plant product of claim 25, wherein the product is a low acrylamide product.

28. A method for producing a commodity plant product, the method comprising processing the plant of any one of claims 1-12, or a part thereof, to obtain the product.

29. The method of claim 28, wherein the commodity plant product is grain, flour, a baked good, cereal, pasta, a beverage, livestock feed, biofuel, straw, a construction material, or starch.

30. A primer pair having the nucleotide sequences of SEQ ID NOs: 14 and 15, SEQ ID NOs: 39 and 40, SEQ ID NOs: 41 and 42, or SEQ ID NOs: 43 and 44.

31. A method of determining the presence of a loss-of-function mutation in an ASN-A2 gene, an ASN-B2 gene, or an ASN-D2 gene in a wheat plant comprising:
assaying a nucleic acid sample from the wheat plant with at least one primer pair of claim 30.

32. A guide RNA for editing an ASN2 gene comprising the nucleotide sequence of SEQ ID NO: 30.

33. A vector encoding the guide RNA of claim 32.

34. A wheat plant cell comprising a Cas9 nuclease and the guide RNA of claim 32.

35. A nucleic acid molecule comprising the nucleotide sequence of SEQ ID NO: 52, 53, 54, 55, 56, 57, 58, or 59.

36. A wheat plant or plant cell comprising at least one nucleic acid molecule of claim 35.


SEQUENCE LISTING (83 sequences)

SEQ ID NO: 1 - DNA, 3800 bp, Triticum aestivum
  source 1..3800: mol_type = genomic DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccggcgaccagccgctctacaacgaggacaagtccatcgccgttgccgtcaacggggaggtctacaaccatgaggagcttcgggcacggctctccggacacaggttccggaccggcagtgactgcgaggtcatcgcccatctggtcagtcacccaacacacaccagccttcttcttctacttgatatataaattcaatcgtatgaaacaataagtttggtcaatccgtagcttctagcatacaaaaaaaagatgatccagataatctctatacatcctaatttgaggttaatttcatatagtaatcagcgaaattaaagttaagtgctagaccattatatctctaatttgttgattaaaattttaataaaaggaagatcttggcgaatatacataggtggacatggttccacctgcacctgaatgacaattaaaattttaaaatatatcagagaattaaacaattctgaattttttaaacaaatatgatccggtgttctatatatatccaccgtggactttcatgaaaaaataacatccattgtgtaccgggataaaatgaaaaaaaacaggtgctctaaaatggtttgaaaatgctcggctcgttaagcttaacgacaagaaaactataatcggctcggccgtaagtacttgacatgcatcaccccttttgctccagttactctgtgttttttatgcaagaagaaagtgaccacacatgaggaggtgcactagttattgtctcctatgggttgagttgggtcaattagatagagttgatgggttggtgtgccatgaaatgttgccgcttaacttaagaatatgtgttgtgttgtattaacgagctgagctgctcgtgaaattcgttaactcctcattaagcttattaagtttaacgagctgaaactagcgtgattggccatgagctacgagtttaatgacccgagctactgagcgttcattatgcttgcgagctacgaggttttggtccacccctaaccacgggtgtgatttcttggtgggtaggacactcgaccaggtttgttttaaaaaatcttttttttttgctaattttcctgatttctctaatcatgtgagtgcgtgcacaccagagccagaactccatttttagattcttgcatgattaaaaagaaagtagaactttggaaaggtggagaaatgtacagctcttagtggcattctctttgtttagcatgacctgactattttctcccttttgatatatagtatgaggaatacggagaaagcttcattgacatgttggatggtgttttctccttcgtgttacttgacgcacgagataacagcttcattgctgctcgtgatgccattggtgtcacgcctctctacattggctggggaattgatggtatagtatcaaaagtgcttcaggaatcttttcatgaaacaaaactatcatttgctctgtgttaatatacaccttgtgaagtagtggttgtcattatcaacaacatgcacactgtggaaaattgtcaggttcagtgtggatatcttcggagatgaaaggactaaacgatgattgtgagcacttcgagatcttcccgcctggtaatctttactccagcaaagagaagtccttcaagagatggtataaccctccttggttctctgaggtcatcccctcggttccctatgacccactgcgtctcagatcggcatttgaaaaggtcacaaactatacgcacctcaactaaccattatgtgctagtatctttgaaaacagatatgtaaccaaattaaggagtcatcgccatgattcttctaactgcatcttctgcattcaggctgttatcaagaggctcatgacagatgttccatttggcgtcctcctctccggtggtctcgactcatcattggtggctgctgtcgcagcccgtcatttcgctgggacgaaggctgcaaagcgctggggaactaggctccactccttctgtgtggggcttgaggtctgaaactgaaagggaacatgtgcatgtttattgaagtttcaaagaaataggacttgcatccatgttatcttaatttatattgtagggatcaccagatctcaaggctgcaaaggaggtcgcggatcacctgggtaccgtgcaccacgagttcaacttcacagttcaggtacgatattactactaagaggcataacgatcgtagcttatttgttgatcgcaatacgcaaccagtaatgttaattgcataaattgctataggatggcatcgatgcaattgaagatgtgatataccacattgaaacatatgatgtgacgacgatcagggcaagcacactgatgttccagatgtcacgcaagatcaaggcgcttggagtcaagatggtcatctccggtgagggtgccgatgagatcttcggagggtacttgtatttccacaaggcccctaacaaggaggagttccaccaggaaacatgtcggaaggtatttaaaatgcaaacattgcagaggaacagaaagaataggacttatccataatagcactgtgctctgacatgaattgttctgcagataaaagctctccatcagtacgattgcttgagggccaacaaagcaacatctgcatggggccttgaggttcgtgtgccattcttggacaaggagttcatcaatgaggctatgagcatagatcccgaatggaagatggtaagcacaaaatatatatttcacggggaaagcccaaaacatttcaaaagaatacacgtcgaaaaaacatttcaaacgaatgggaagttggaaagaatggactgcgttgttttctaacttttaatcacacagatccggcctgatcttggaagaattgagaaatggatactgaggaaagcgttcgatgatgaggagcgacccttcctgccgaaggtaacatttttatacacttcagtcagatccagctggagtagtggagttattaacatgttctttttttatacatgtagcatattctgtacaggcagaaggagcagtttagtgatggtgttgggtatagctggattgatggcctgaaggatcatgcagcctcaaatgtaaatgaacaatttttttattttaaagaattctttatgatgtttgttttgacaaaactaatacattccctgatagaaagatgtgaccacaatatctgacctatatgaaatgaaatcactgaatgcaggtgagtgataagatgatgtccaatgcaaagttcatctacccacacaacaccccaacaactaaagaggcctactattacaggatgatctttgagaggtacttcccccaggtgagtgactaaatagaacaggttgaagaacaacatgaatagttcaataccattgtagcaatgttaatggaagcaatgttggttgcttgatcttgtttcagagctcggcgatcctcacggtgccaggcgggccaagcgtggcgtgcagcacagccaaggctatagagtgggatgcccaatggtctgggaacctggacccctctgggagagcagcgcttggagtccatctctcagcctacgagcaggacacggtcgctgtgggaggtagcaacaagcctggggtgatgaacaccgtggtacctggtgttgccattgagacttga

SEQ ID NO: 2 - DNA, 2819 bp, Triticum aestivum
  source 1..2819: mol_type = genomic DNA
atgtgtggcatactggccgtgctggggtgcggcgacgagtcgcaggggaagagggtccacgtcctcgagctctcgcgcaggctcaagcaccgcggcccggactggagcggcctgcaccaggtcgccgacaactacctctgccaccagcgcctcgccatcatcgacccggcctccggcgaccagccgctctacaacgaggacaagtccgtcgccgtcgccgtcaacggggagatctacaaccatgaggagctccgggcacggctctccggccacagcttccggaccggcagtgactgcgaggtcatcgcccatttggtcagtcacccaccacacacacaccaacattcttcttctgcttgacatattaagtacttcactacaaatctgacctcaaattatttttaccttttaccatggcaaattgaactttttttagtgccattttttttgtttagcatgacctgactattttctcccttttgacatatagtatgaggaatacggagaaagctccattgagatgttggatggtgttttctccttcgtgttgcttgacgcaagagatcacagcttcatcgctgctcgtgatgccattggtgtcacgcctctctacattggctggggaattgatggtataatatcaaaagtgctttcatgaaacagaactatcatttgcactgtgttaatatacaccttgtgaagtagtggctgtcatcatcatcaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcaattccctatgacccactgcgtctcagatcggcatttgaaaaggtcacaaaccacacttgcctcaactaaccgttatgtgcatctttgaaaaacagagatgtaaccaaatttaggagtcaagagtcaactaaccatgattcttctgactgcatcttctgcatccaggctgtcatcaagaggctcatgacagatgttccatttggcgtcctcctctccggtggtctcgactcatcactggtggcagctgtcgcagcccgtcattttgcagggacaaaggctgcaaaacgttggggaactaggctccactccttctgtgttgggcttgaggtctgaaacttaaaggaacatgtgcatgtttatcgaagtttcaaagtaataccacttgcaaccatgctgttatcttaatttatattgtagggatcaccggatctcaaggctgcaaaggaggtcgcggatcatctgggtactgtgcaccatgagttcaacttcacagttcaggcatgatattaccactaagaagcataacgatcgtcgcttatttgttgatcacaatacagaatcactaattttaatagcacatattgctataggacggcatcgatgcaattgaagatgtgatatatcacattgaaacatatgacgtgacgacgatcagggcaagcacactgatgttccagatgtcacgcaagatcaaggcgcttggtgtcaagatggtcatctctggtgagggcgccgatgaaatctttggagggtacttgtatttccacaaggcccctaacaaggaggagttccaccaggaaacatgtcggaaggtctttaagatacaaacgtttcagaggagcagaaacaacatataggaccaacacataacactctgctctgacataaattgttctgcagatcaaagctctccatcagtacgattgcttgagggccaacaaagcaacatctgcatggggccttgaggttcgtgtgccattcttggacaaggagttcatcaatgaggctatgagcatagaccccgaatggaagatggtaagcacaaaacatttcatgagaatgggaaattgaaaatagtgcactgcactgttttctaaatttaatcacacagatccggcctgatcttggaagaattgaaaaatggatattgaggaaagcatttgatgacgaggagaaaccgttcctgccgaaggtaacattttttagactcagcagtcagatccagctggagtagtggagttattaacatgttcttttgtgtgatctatgtagcacattctgtacaggcagaaggagcagtttagtgatggtgttgggtatagctggattgatggcctgaaggatcatgcggcatcaaatgtaaatgaacaattttattcattttaaagaatctttacgatgtttgtttcgacaaaactaatgcattccttgaaagaaagacgcgaccacaatatctgacctatatgacatgaaatcactgaatgcaggtgagtgataagatgatgtccaatgcaaagttcatctacccacacaacaccccaacgactaaagaggcttactattacaggatgatctttgagaggtacttcccccaggtgagtcaataaatagaacaggttgaagaaaacaacatgaatagttcaataccattgtagcaatgttaatggaagcaatgtcggttgctcgatcttgtttcagagctcggcgatcctcacggtgccaggcgggccaagcgtggcgtgcagcacagccaaggctatagagtgggatgcccagtggtctgggaacctggacccctccgggagagcagcgcttggagtccatctctcggcctacgagcaggacacggtagcagtgggaggtagcaacaagcctggggtgatgaggaccgcggtgcctggtgttgccatcgagacctga

SEQ ID NO: 3 - DNA, 4057 bp, Triticum aestivum
  source 1..4057: mol_type = genomic DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccggcgaccagccgctctacaacgaggacaagtccgtctccgtcgccgtgaacggggagatttacaaccatgaggagctccgggcacggctctccggccacaggttccggaccggcagtgactgcgaggtcatcgcccatctggtcagtcacccaccgcacacaccaccattctttcttctgcttgatactacgtatatactagtaaatgagcacgtgctgcatgttgatattagggaggaaattaactgcatgttaatattaggtaggatatcaattacgtgttaattatgaaattataggtaggatatcagttacgtgttaattatgagattaatatagatgttgatatttggtatgctatcaattgcacgctcaacatgttgagagctcatcactagagcaatctaggtcgttgaattgacatggtttgatggccgagattaattgaatctgccaatttgggtctttttatattgaaatataggtatagataaattcaacagtataaaacaataagtttggtgaatccgtagcttctagcacagaaacacaaagataatccggataatctctatacatcctaatttgaggttaatttcgtatattaatcagcgaaataaaagttaagtgctagaccattatatctctaatctgttgattacaattttaataaaaggaagatcttggcgaatatacatcggtgcacctgaatgacgagtaagattttaaaatataacaaagaattaaacaattctgattttttaaacaaatatgatgagtgttctatatccactgtggacttccatgaaaaaatgacatccattgtgtaccgggataaaatgaaaaaaaaacatatgctctaaaatggtttgaaaatgctcggctcgttaagcttaacgacaagaaaactatgatcggctcgtaagtacttgacatgcaataaccccttttgctccagttactgtgtgttttttatgcaagaagaaagtgaccagatgtggaggtacactagttcttgtctcctatgggttgagttgggtcaattagatggagttgatgggttggtgtgccatgaaatgttgccgcttaacttaagaatatgtgttgtgttgtattaacgagctgagctgctcgtgaaattcgttaactcctcattaagctcattaagggaaaattatgctcattaagtttaacgagctgaaactagcggtgattggccctgagctatgctcattaagtttaatgacccgagctactgagcattcattatgcttgcgagctatgaggttttggtccatccctaaccacgggtgtgatttcttggtgggtaggacactcgatcaggtttgtttaaaaaaatcattttttttgctatttttcctgatttctctaatcatgtaagtgcgtgcacaccagagccagaactccatttttagattcttgcatgattaaaaagaaagtagaactttggaaaggtggagaaatgtacagctcttagtggcattctttttgtttagcatgacctgactattttctcccttttgatatatagtatgaggaatacggagaaagattcattgacatgttggatggtgttttctccttcgtgttgcttgacgcacgagataagagcttcattgctgctcgtgatgccattggtgtcacgcctctctacattggctggggaattgatggtatagcatcaaaagtggttgaggaatcttttcatgaagcaaacctatcatttgctctgtgttactaaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggttgcaaactatacccgactcaactaaccacactaatatcttggaagaacagagaagtgaccaaatttaggactcatggtcatgattcttctaaattcatgctcttcattcaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgaggtctggaactgaaaggaacatgtgcatgtttattgaagtttcaaagtaatagcacctgcaaccatgctgttatcttaatttatattgtagggctcaccggatctcaaggctgcaaaggaggtcgcggatcacctgggtaccgtgcaccatgagttcaacttcacagttcaggtacgatattactactaagaggcataacgatcgtagcttatttgttgatcacaatacgcaaccagtaatgttaattgcataaattgctataggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaaggtatttaaagtagaagcgtttcagaggagcagaaagaataggacataaccataataacactctgctctgacataaaattgctctacagattaaagctctccatcagtacgattgcttgagggctaacaaagcaacatctgcatggggccttgaggttcgtgtgccattcttggacaaggagttcatcgatgaggctatgagcatagatcccgaatggaagatggtaagcacaaaaaatatatttcacggggaaagcccaaaacatttcgaaagaatacgcgtcaaaaaaatatttcaaacgaatgggaagttagaaagaatggactgcattgttttctaaatttaatgacacagatccggcctgatcttggaagaattgagaaatggatactgaggaaagcgttcgatgatgaggagcaacccttcctgccgaaggtaacatttttatacacttcagtcagatccagttggagtagtggagttattaacaggttctttttttatacatgtagcatattctgtacaggcagaaggagcagtttagtgatggtgttgggtatagctggattgatggcctgaaggatcatgcagcatcaaatgtaaatgaacaatttttttattttgaagaattctttatgatgtttgtttcgacaaaactaatgcattccttgataggaagatgtgactgcaatatctgacctctatgaaatgaaatcactgaatgcaggtgagtgataagatgatgtccaatgcaaagttcatctacccacacaacaccccaacaactaaagaggcctactattacaggatgatcttcgagaggtacttcccccaggtgagtgactaaatagaacaagttgaaaaacaacacgaatagttcaataccattgtagcaatgttgattgctcaatcttgtttcagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 4 - DNA, 1503 bp, Synthetic construct
  source 1..1503: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccggcgaccagccgctctacaacgaggacaagtccatcgccgttgccgtcaacggggaggtctacaaccatgaggagcttcgggcacggctctccggacacaggttccggaccggcagtgactgcgaggtcatcgcccatctgtatgaggaatacggagaaagcttcattgacatgttggatggtgttttctccttcgtgttacttgacgcacgagataacagcttcattgctgctcgtgatgccattggtgtcacgcctctctacattggctggggaattgatggttcagtgtggatatcttcggagatgaaaggactaaacgatgattgtgagcacttcgagatcttcccgcctggtaatctttactccagcaaagagaagtccttcaagagatggtataaccctccttggttctctgaggtcatcccctcggttccctatgacccactgcgtctcagatcggcatttgaaaaggatggcatcgatgcaattgaagatgtgatataccacattgaaacatatgatgtgacgacgatcagggcaagcacactgatgttccagatgtcacgcaagatcaaggcgcttggagtcaagatggtcatctccggtgagggtgccgatgagatcttcggagggtacttgtatttccacaaggcccctaacaaggaggagttccaccaggaaacatgtcggaagataaaagctctccatcagtacgattgcttgagggccaacaaagcaacatctgcatggggccttgaggttcgtgtgccattcttggacaaggagttcatcaatgaggctatgagcatagatcccgaatggaagatgatccggcctgatcttggaagaattgagaaatggatactgaggaaagcgttcgatgatgaggagcgacccttcctgccgaagcatattctgtacaggcagaaggagcagtttagtgatggtgttgggtatagctggattgatggcctgaaggatcatgcagcctcaaatgtgagtgataagatgatgtccaatgcaaagttcatctacccacacaacaccccaacaactaaagaggcctactattacaggatgatctttgagaggtacttcccccagagctcggcgatcctcacggtgccaggcgggccaagcgtggcgtgcagcacagccaaggctatagagtgggatgcccaatggtctgggaacctggacccctctgggagagcagcgcttggagtccatctctcagcctacgagcaggacacggtcgctgtgggaggtagcaacaagcctggggtgatgaacaccgtggtacctggtgttgccattgagacttga

SEQ ID NO: 5 - DNA, 1746 bp, Synthetic construct
  source 1..1746: mol_type = other DNA
atgtgtggcatactggccgtgctggggtgcggcgacgagtcgcaggggaagagggtccacgtcctcgagctctcgcgcaggctcaagcaccgcggcccggactggagcggcctgcaccaggtcgccgacaactacctctgccaccagcgcctcgccatcatcgacccggcctccggcgaccagccgctctacaacgaggacaagtccgtcgccgtcgccgtcaacggggagatctacaaccatgaggagctccgggcacggctctccggccacagcttccggaccggcagtgactgcgaggtcatcgcccatttgtatgaggaatacggagaaagctccattgagatgttggatggtgttttctccttcgtgttgcttgacgcaagagatcacagcttcatcgctgctcgtgatgccattggtgtcacgcctctctacattggctggggaattgatggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcaattccctatgacccactgcgtctcagatcggcatttgaaaaggctgtcatcaagaggctcatgacagatgttccatttggcgtcctcctctccggtggtctcgactcatcactggtggcagctgtcgcagcccgtcattttgcagggacaaaggctgcaaaacgttggggaactaggctccactccttctgtgttgggcttgagggatcaccggatctcaaggctgcaaaggaggtcgcggatcatctgggtactgtgcaccatgagttcaacttcacagttcaggacggcatcgatgcaattgaagatgtgatatatcacattgaaacatatgacgtgacgacgatcagggcaagcacactgatgttccagatgtcacgcaagatcaaggcgcttggtgtcaagatggtcatctctggtgagggcgccgatgaaatctttggagggtacttgtatttccacaaggcccctaacaaggaggagttccaccaggaaacatgtcggaagatcaaagctctccatcagtacgattgcttgagggccaacaaagcaacatctgcatggggccttgaggttcgtgtgccattcttggacaaggagttcatcaatgaggctatgagcatagaccccgaatggaagatgatccggcctgatcttggaagaattgaaaaatggatattgaggaaagcatttgatgacgaggagaaaccgttcctgccgaagcacattctgtacaggcagaaggagcagtttagtgatggtgttgggtatagctggattgatggcctgaaggatcatgcggcatcaaatgtgagtgataagatgatgtccaatgcaaagttcatctacccacacaacaccccaacgactaaagaggcttactattacaggatgatctttgagaggtacttcccccagagctcggcgatcctcacggtgccaggcgggccaagcgtggcgtgcagcacagccaaggctatagagtgggatgcccagtggtctgggaacctggacccctccgggagagcagcgcttggagtccatctctcggcctacgagcaggacacggtagcagtgggaggtagcaacaagcctggggtgatgaggaccgcggtgcctggtgttgccatcgagacctga

SEQ ID NO: 6 - DNA, 1746 bp, Synthetic construct
  source 1..1746: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccggcgaccagccgctctacaacgaggacaagtccgtctccgtcgccgtgaacggggagatttacaaccatgaggagctccgggcacggctctccggccacaggttccggaccggcagtgactgcgaggtcatcgcccatctgtatgaggaatacggagaaagattcattgacatgttggatggtgttttctccttcgtgttgcttgacgcacgagataagagcttcattgctgctcgtgatgccattggtgtcacgcctctctacattggctggggaattgatggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgagggctcaccggatctcaaggctgcaaaggaggtcgcggatcacctgggtaccgtgcaccatgagttcaacttcacagttcaggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaagattaaagctctccatcagtacgattgcttgagggctaacaaagcaacatctgcatggggccttgaggttcgtgtgccattcttggacaaggagttcatcgatgaggctatgagcatagatcccgaatggaagatgatccggcctgatcttggaagaattgagaaatggatactgaggaaagcgttcgatgatgaggagcaacccttcctgccgaagcatattctgtacaggcagaaggagcagtttagtgatggtgttgggtatagctggattgatggcctgaaggatcatgcagcatcaaatgtgagtgataagatgatgtccaatgcaaagttcatctacccacacaacaccccaacaactaaagaggcctactattacaggatgatcttcgagaggtacttcccccagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 7 - protein, 500 aa, Triticum aestivum
MCGILAVLGCGDESQGKRVHVLELSRRLKHRGPDWSGLHQVADNYLCHQRLAIIDPASGDQPLYNEDKSIAVAVNGEVYNHEELRARLSGHRFRTGSDCEVIAHLYEEYGESFIDMLDGVFSFVLLDARDNSFIAARDAIGVTPLYIGWGIDGSVWISSEMKGLNDDCEHFEIFPPGNLYSSKEKSFKRWYNPPWFSEVIPSVPYDPLRLRSAFEKDGIDAIEDVIYHIETYDVTTIRASTLMFQMSRKIKALGVKMVISGEGADEIFGGYLYFHKAPNKEEFHQETCRKIKALHQYDCLRANKATSAWGLEVRVPFLDKEFINEAMSIDPEWKMIRPDLGRIEKWILRKAFDDEERPFLPKHILYRQKEQFSDGVGYSWIDGLKDHAASNVSDKMMSNAKFIYPHNTPTTKEAYYYRMIFERYFPQSSAILTVPGGPSVACSTAKAIEWDAQWSGNLDPSGRAALGVHLSAYEQDTVAVGGSNKPGVMNTVVPGVAIET

SEQ ID NO: 8 - protein, 581 aa, Triticum aestivum
MCGILAVLGCGDESQGKRVHVLELSRRLKHRGPDWSGLHQVADNYLCHQRLAIIDPASGDQPLYNEDKSVAVAVNGEIYNHEELRARLSGHSFRTGSDCEVIAHLYEEYGESSIEMLDGVFSFVLLDARDHSFIAARDAIGVTPLYIGWGIDGSVWISSEMKGLNDDCEHFEIFPPGNLYSSKEKCFKRWYNPPWFSEVIPSIPYDPLRLRSAFEKAVIKRLMTDVPFGVLLSGGLDSSLVAAVAARHFAGTKAAKRWGTRLHSFCVGLEGSPDLKAAKEVADHLGTVHHEFNFTVQDGIDAIEDVIYHIETYDVTTIRASTLMFQMSRKIKALGVKMVISGEGADEIFGGYLYFHKAPNKEEFHQETCRKIKALHQYDCLRANKATSAWGLEVRVPFLDKEFINEAMSIDPEWKMIRPDLGRIEKWILRKAFDDEEKPFLPKHILYRQKEQFSDGVGYSWIDGLKDHAASNVSDKMMSNAKFIYPHNTPTTKEAYYYRMIFERYFPQSSAILTVPGGPSVACSTAKAIEWDAQWSGNLDPSGRAALGVHLSAYEQDTVAVGGSNKPGVMRTAVPGVAIET

SEQ ID NO: 9 - protein, 581 aa, Triticum aestivum
MCGILAVLGCGDESQGKRVHVLELSRRLKHRGPDWSGLHQVADNYLCHQRLAIIDPASGDQPLYNEDKSVSVAVNGEIYNHEELRARLSGHRFRTGSDCEVIAHLYEEYGERFIDMLDGVFSFVLLDARDKSFIAARDAIGVTPLYIGWGIDGSVWISSEMKGLNDDCEHFEIFPPGNLYSSKEKCFKRWYNPPWFSEVIPSVPYDPLRLRSAFEKAVIKRLMTDVPFGVLLSGGLDSSLVAAVAARHFAGTKAAKRWGTRLHSFCVGLEGSPDLKAAKEVADHLGTVHHEFNFTVQDGIDAIEDVIYHIETYDVTTIRASTLMFQMSRKIKALGVKMVISGEGADEIFGGYLYFHKAPNKEEFHQETCRKIKALHQYDCLRANKATSAWGLEVRVPFLDKEFIDEAMSIDPEWKMIRPDLGRIEKWILRKAFDDEEQPFLPKHILYRQKEQFSDGVGYSWIDGLKDHAASNVSDKMMSNAKFIYPHNTPTTKEAYYYRMIFERYFPQSSAILTVPGGPSVACSTAKAIEWDAQWSGNLDPSGRAALGVHLSAYEQDTVAVGGSNKHGVMKTVVPGVAIET

SEQ ID NO: 10 - DNA, 20 bp, Synthetic construct
  source 1..20: mol_type = other DNA
agcaagccttcaccatcatt

SEQ ID NO: 11 - DNA, 21 bp, Synthetic construct
  source 1..21: mol_type = other DNA
gatgtaggcatgtcaacgaga

SEQ ID NO: 12 - DNA, 20 bp, Synthetic construct
  source 1..20: mol_type = other DNA
aacaagcctggggtgatgag

SEQ ID NO: 13 - DNA, 25 bp, Synthetic construct
  source 1..25: mol_type = other DNA
ttgtctcaaaaagaaaaagaacttg

SEQ ID NO: 14 - DNA, 20 bp, Synthetic construct
  source 1..20: mol_type = other DNA
aagaagaaagtgaccacaca

SEQ ID NO: 15 - DNA, 24 bp, Synthetic construct
  source 1..24: mol_type = other DNA
ttttcaaagatactagcacataat

SEQ ID NO: 16 - DNA, 45 bp, Synthetic construct
  source 1..45: mol_type = other DNA
gaaggtgaccaagttcatgctagtcctttcatctccgaagatatc

SEQ ID NO: 17 - DNA, 45 bp, Synthetic construct
  source 1..45: mol_type = other DNA
gaaggtcggagtcaacggattagtcctttcatctccgaagatatt

SEQ ID NO: 18 - DNA, 20 bp, Synthetic construct
  source 1..20: mol_type = other DNA
acaacatgcacactgtggaa

SEQ ID NO: 19 - DNA, 41 bp, Synthetic construct
  source 1..41: mol_type = other DNA
gaaggtgaccaagttcatgctacgcctctctacattggctg

SEQ ID NO: 20 - DNA, 41 bp, Synthetic construct
  source 1..41: mol_type = other DNA
gaaggtcggagtcaacggattacgcctctctacattggcta

SEQ ID NO: 21 - DNA, 23 bp, Synthetic construct
  source 1..23: mol_type = other DNA
catgaaaagattcctgaagcact

SEQ ID NO: 22 - DNA, 20 bp, Synthetic construct
  source 1..20: mol_type = other DNA
tcaacggggaggtctacaac

SEQ ID NO: 23 - DNA, 22 bp, Synthetic construct
  source 1..22: mol_type = other DNA
gcaatgaagctgttatctcgtg

SEQ ID NO: 24 - DNA, 22 bp, Synthetic construct
  source 1..22: mol_type = other DNA
gtcaacggggagatctacaacc

SEQ ID NO: 25 - DNA, 22 bp, Synthetic construct
  source 1..22: mol_type = other DNA
gcgatgaagctgtgatctcttg

SEQ ID NO: 26 - DNA, 21 bp, Synthetic construct
  source 1..21: mol_type = other DNA
gtgaacggggagatttacaac

SEQ ID NO: 27 - DNA, 22 bp, Synthetic construct
  source 1..22: mol_type = other DNA
gcaatgaagctcttatctcgtg

SEQ ID NO: 28 - DNA, 24 bp, Synthetic construct
  source 1..24: mol_type = other DNA
cagagtcgagcacaataccagttg

SEQ ID NO: 29 - DNA, 20 bp, Synthetic construct
  source 1..20: mol_type = other DNA
accttcagttgcccagcaat

SEQ ID NO: 30 - DNA, 20 bp, Synthetic construct
  source 1..20: mol_type = other DNA
gtagagcggctggtcgccgg

SEQ ID NO: 31 - DNA, 23 bp, Triticum aestivum
  source 1..23: mol_type = genomic DNA
cctctggcgaccagcccctctac

SEQ ID NO: 32 - DNA, 23 bp, Triticum aestivum
  source 1..23: mol_type = genomic DNA
cctctggcgaccagccgctctac

SEQ ID NO: 33 - DNA, 23 bp, Triticum aestivum
  source 1..23: mol_type = genomic DNA
cctccggcgaccagccgctctac

SEQ ID NO: 34 - DNA, 23 bp, Triticum aestivum
  source 1..23: mol_type = genomic DNA
catctggagaccagccattgtac

SEQ ID NO: 35 - DNA, 23 bp, Triticum aestivum
  source 1..23: mol_type = genomic DNA
catctggagaccagccactgtac

SEQ ID NO: 36 - DNA, 23 bp, Triticum aestivum
  source 1..23: mol_type = genomic DNA
tctccggcgaccagcccctctac

SEQ ID NO: 37 - DNA, 23 bp, Triticum aestivum
  source 1..23: mol_type = genomic DNA
tctccggtgaccagcccctctac

SEQ ID NO: 38 - DNA, 20 bp, Synthetic construct
  source 1..20: mol_type = other DNA
ccggcgaccagccgctctac

SEQ ID NO: 39 - DNA, 20 bp, Synthetic construct
  source 1..20: mol_type = other DNA
catggacgacgtgtagccct

SEQ ID NO: 40 - DNA, 26 bp, Synthetic construct
  source 1..26: mol_type = other DNA
ttgtatgctagaagctacggattgac

SEQ ID NO: 41 - DNA, 22 bp, Synthetic construct
  source 1..22: mol_type = other DNA
gagaggaaagcagagcagtctt

SEQ ID NO: 42 - DNA, 20 bp, Synthetic construct
  source 1..20: mol_type = other DNA
accttccgacatgtttcctg

SEQ ID NO: 43 - DNA, 20 bp, Synthetic construct
  source 1..20: mol_type = other DNA
tccagaggagcagcataacc

SEQ ID NO: 44 - DNA, 18 bp, Synthetic construct
  source 1..18: mol_type = other DNA
aagaatggtggtgtgtgc

SEQ ID NO: 45 - DNA, 316 bp, Synthetic construct
  source 1..316: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccgtgcgaccagccgctctacaacgaggacaagtccatcgccgttgccgtcaacggggaggtctacaaccatgaggagcttcgggcacggctctccggacacaggttccggaccggcagtgactgcgaggtcatcgcccatctg

SEQ ID NO: 46 - DNA, 314 bp, Synthetic construct
  source 1..314: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgggaccagccgctctacaacgaggacaagtccgtctccgtcgccgtgaacggggagatttacaaccatgaggagctccgggcacggctctccggccacaggttccggaccggcagtgactgcgaggtcatcgcccatctg

SEQ ID NO: 47 - DNA, 316 bp, Synthetic construct
  source 1..316: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccgagcgaccagccgctctacaacgaggacaagtccatcgccgttgccgtcaacggggaggtctacaaccatgaggagcttcgggcacggctctccggacacaggttccggaccggcagtgactgcgaggtcatcgcccatctg

SEQ ID NO: 48 - DNA, 316 bp, Synthetic construct
  source 1..316: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgagcgaccagccgctctacaacgaggacaagtccgtctccgtcgccgtgaacggggagatttacaaccatgaggagctccgggcacggctctccggccacaggttccggaccggcagtgactgcgaggtcatcgcccatctg

SEQ ID NO: 49 - DNA, 316 bp, Synthetic construct
  source 1..316: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgtgcgaccagccgctctacaacgaggacaagtccgtctccgtcgccgtgaacggggagatttacaaccatgaggagctccgggcacggctctccggccacaggttccggaccggcagtgactgcgaggtcatcgcccatctg

SEQ ID NO: 50 - DNA, 316 bp, Synthetic construct
  source 1..316: mol_type = other DNA
atgtgtggcatactggccgtgctggggtgcggcgacgagtcgcaggggaagagggtccacgtcctcgagctctcgcgcaggctcaagcaccgcggcccggactggagcggcctgcaccaggtcgccgacaactacctctgccaccagcgcctcgccatcatcgacccggcctccgagcgaccagccgctctacaacgaggacaagtccgtcgccgtcgccgtcaacggggagatctacaaccatgaggagctccgggcacggctctccggccacagcttccggaccggcagtgactgcgaggtcatcgcccatttg

SEQ ID NO: 51 - DNA, 316 bp, Synthetic construct
  source 1..316: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgcgcgaccagccgctctacaacgaggacaagtccgtctccgtcgccgtgaacggggagatttacaaccatgaggagctccgggcacggctctccggccacaggttccggaccggcagtgactgcgaggtcatcgcccatctg

SEQ ID NO: 52 - DNA, 3801 bp, Synthetic construct
  source 1..3801: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccgtgcgaccagccgctctacaacgaggacaagtccatcgccgttgccgtcaacggggaggtctacaaccatgaggagcttcgggcacggctctccggacacaggttccggaccggcagtgactgcgaggtcatcgcccatctggtcagtcacccaacacacaccagccttcttcttctacttgatatataaattcaatcgtatgaaacaataagtttggtcaatccgtagcttctagcatacaaaaaaaagatgatccagataatctctatacatcctaatttgaggttaatttcatatagtaatcagcgaaattaaagttaagtgctagaccattatatctctaatttgttgattaaaattttaataaaaggaagatcttggcgaatatacataggtggacatggttccacctgcacctgaatgacaattaaaattttaaaatatatcagagaattaaacaattctgaattttttaaacaaatatgatccggtgttctatatatatccaccgtggactttcatgaaaaaataacatccattgtgtaccgggataaaatgaaaaaaaacaggtgctctaaaatggtttgaaaatgctcggctcgttaagcttaacgacaagaaaactataatcggctcggccgtaagtacttgacatgcatcaccccttttgctccagttactctgtgttttttatgcaagaagaaagtgaccacacatgaggaggtgcactagttattgtctcctatgggttgagttgggtcaattagatagagttgatgggttggtgtgccatgaaatgttgccgcttaacttaagaatatgtgttgtgttgtattaacgagctgagctgctcgtgaaattcgttaactcctcattaagcttattaagtttaacgagctgaaactagcgtgattggccatgagctacgagtttaatgacccgagctactgagcgttcattatgcttgcgagctacgaggttttggtccacccctaaccacgggtgtgatttcttggtgggtaggacactcgaccaggtttgttttaaaaaatcttttttttttgctaattttcctgatttctctaatcatgtgagtgcgtgcacaccagagccagaactccatttttagattcttgcatgattaaaaagaaagtagaactttggaaaggtggagaaatgtacagctcttagtggcattctctttgtttagcatgacctgactattttctcccttttgatatatagtatgaggaatacggagaaagcttcattgacatgttggatggtgttttctccttcgtgttacttgacgcacgagataacagcttcattgctgctcgtgatgccattggtgtcacgcctctctacattggctggggaattgatggtatagtatcaaaagtgcttcaggaatcttttcatgaaacaaaactatcatttgctctgtgttaatatacaccttgtgaagtagtggttgtcattatcaacaacatgcacactgtggaaaattgtcaggttcagtgtggatatcttcggagatgaaaggactaaacgatgattgtgagcacttcgagatcttcccgcctggtaatctttactccagcaaagagaagtccttcaagagatggtataaccctccttggttctctgaggtcatcccctcggttccctatgacccactgcgtctcagatcggcatttgaaaaggtcacaaactatacgcacctcaactaaccattatgtgctagtatctttgaaaacagatatgtaaccaaattaaggagtcatcgccatgattcttctaactgcatcttctgcattcaggctgttatcaagaggctcatgacagatgttccatttggcgtcctcctctccggtggtctcgactcatcattggtggctgctgtcgcagcccgtcatttcgctgggacgaaggctgcaaagcgctggggaactaggctccactccttctgtgtggggcttgaggtctgaaactgaaagggaacatgtgcatgtttattgaagtttcaaagaaataggacttgcatccatgttatcttaatttatattgtagggatcaccagatctcaaggctgcaaaggaggtcgcggatcacctgggtaccgtgcaccacgagttcaacttcacagttcaggtacgatattactactaagaggcataacgatcgtagcttatttgttgatcgcaatacgcaaccagtaatgttaattgcataaattgctataggatggcatcgatgcaattgaagatgtgatataccacattgaaacatatgatgtgacgacgatcagggcaagcacactgatgttccagatgtcacgcaagatcaaggcgcttggagtcaagatggtcatctccggtgagggtgccgatgagatcttcggagggtacttgtatttccacaaggcccctaacaaggaggagttccaccaggaaacatgtcggaaggtatttaaaatgcaaacattgcagaggaacagaaagaataggacttatccataatagcactgtgctctgacatgaattgttctgcagataaaagctctccatcagtacgattgcttgagggccaacaaagcaacatctgcatggggccttgaggttcgtgtgccattcttggacaaggagttcatcaatgaggctatgagcatagatcccgaatggaagatggtaagcacaaaatatatatttcacggggaaagcccaaaacatttcaaaagaatacacgtcgaaaaaacatttcaaacgaatgggaagttggaaagaatggactgcgttgttttctaacttttaatcacacagatccggcctgatcttggaagaattgagaaatggatactgaggaaagcgttcgatgatgaggagcgacccttcctgccgaaggtaacatttttatacacttcagtcagatccagctggagtagtggagttattaacatgttctttttttatacatgtagcatattctgtacaggcagaaggagcagtttagtgatggtgttgggtatagctggattgatggcctgaaggatcatgcagcctcaaatgtaaatgaacaatttttttattttaaagaattctttatgatgtttgttttgacaaaactaatacattccctgatagaaagatgtgaccacaatatctgacctatatgaaatgaaatcactgaatgcaggtgagtgataagatgatgtccaatgcaaagttcatctacccacacaacaccccaacaactaaagaggcctactattacaggatgatctttgagaggtacttcccccaggtgagtgactaaatagaacaggttgaagaacaacatgaatagttcaataccattgtagcaatgttaatggaagcaatgttggttgcttgatcttgtttcagagctcggcgatcctcacggtgccaggcgggccaagcgtggcgtgcagcacagccaaggctatagagtgggatgcccaatggtctgggaacctggacccctctgggagagcagcgcttggagtccatctctcagcctacgagcaggacacggtcgctgtgggaggtagcaacaagcctggggtgatgaacaccgtggtacctggtgttgccattgagacttga

SEQ ID NO: 53 - DNA, 4056 bp, Synthetic construct
  source 1..4056: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgggaccagccgctctacaacgaggacaagtccgtctccgtcgccgtgaacggggagatttacaaccatgaggagctccgggcacggctctccggccacaggttccggaccggcagtgactgcgaggtcatcgcccatctggtcagtcacccaccgcacacaccaccattctttcttctgcttgatactacgtatatactagtaaatgagcacgtgctgcatgttgatattagggaggaaattaactgcatgttaatattaggtaggatatcaattacgtgttaattatgaaattataggtaggatatcagttacgtgttaattatgagattaatatagatgttgatatttggtatgctatcaattgcacgctcaacatgttgagagctcatcactagagcaatctaggtcgttgaattgacatggtttgatggccgagattaattgaatctgccaatttgggtctttttatattgaaatataggtatagataaattcaacagtataaaacaataagtttggtgaatccgtagcttctagcacagaaacacaaagataatccggataatctctatacatcctaatttgaggttaatttcgtatattaatcagcgaaataaaagttaagtgctagaccattatatctctaatctgttgattacaattttaataaaaggaagatcttggcgaatatacatcggtgcacctgaatgacgagtaagattttaaaatataacaaagaattaaacaattctgattttttaaacaaatatgatgagtgttctatatccactgtggacttccatgaaaaaatgacatccattgtgtaccgggataaaatgaaaaaaaaacatatgctctaaaatggtttgaaaatgctcggctcgttaagcttaacgacaagaaaactatgatcggctcgtaagtacttgacatgcaataaccccttttgctccagttactgtgtgttttttatgcaagaagaaagtgaccagatgtggaggtacactagttcttgtctcctatgggttgagttgggtcaattagatggagttgatgggttggtgtgccatgaaatgttgccgcttaacttaagaatatgtgttgtgttgtattaacgagctgagctgctcgtgaaattcgttaactcctcattaagctcattaagggaaaattatgctcattaagtttaacgagctgaaactagcggtgattggccctgagctatgctcattaagtttaatgacccgagctactgagcattcattatgcttgcgagctatgaggttttggtccatccctaaccacgggtgtgatttcttggtgggtaggacactcgatcaggtttgtttaaaaaaatcattttttttgctatttttcctgatttctctaatcatgtaagtgcgtgcacaccagagccagaactccatttttagattcttgcatgattaaaaagaaagtagaactttggaaaggtggagaaatgtacagctcttagtggcattctttttgtttagcatgacctgactattttctcccttttgatatatagtatgaggaatacggagaaagattcattgacatgttggatggtgttttctccttcgtgttgcttgacgcacgagataagagcttcattgctgctcgtgatgccattggtgtcacgcctctctacattggctggggaattgatggtatagcatcaaaagtggttgaggaatcttttcatgaagcaaacctatcatttgctctgtgttactaaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggttgcaaactatacccgactcaactaaccacactaatatcttggaagaacagagaagtgaccaaatttaggactcatggtcatgattcttctaaattcatgctcttcattcaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgaggtctggaactgaaaggaacatgtgcatgtttattgaagtttcaaagtaatagcacctgcaaccatgctgttatcttaatttatattgtagggctcaccggatctcaaggctgcaaaggaggtcgcggatcacctgggtaccgtgcaccatgagttcaacttcacagttcaggtacgatattactactaagaggcataacgatcgtagcttatttgttgatcacaatacgcaaccagtaatgttaattgcataaattgctataggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaaggtatttaaagtagaagcgtttcagaggagcagaaagaataggacataaccataataacactctgctctgacataaaattgctctacagattaaagctctccatcagtacgattgcttgagggctaacaaagcaacatctgcatggggccttgaggttcgtgtgccattcttggacaaggagttcatcgatgaggctatgagcatagatcccgaatggaagatggtaagcacaaaaaatatatttcacggggaaagcccaaaacatttcgaaagaatacgcgtcaaaaaaatatttcaaacgaatgggaagttagaaagaatggactgcattgttttctaaatttaatgacacagatccggcctgatcttggaagaattgagaaatggatactgaggaaagcgttcgatgatgaggagcaacccttcctgccgaaggtaacatttttatacacttcagtcagatccagttggagtagtggagttattaacaggttctttttttatacatgtagcatattctgtacaggcagaaggagcagtttagtgatggtgttgggtatagctggattgatggcctgaaggatcatgcagcatcaaatgtaaatgaacaatttttttattttgaagaattctttatgatgtttgtttcgacaaaactaatgcattccttgataggaagatgtgactgcaatatctgacctctatgaaatgaaatcactgaatgcaggtgagtgataagatgatgtccaatgcaaagttcatctacccacacaacaccccaacaactaaagaggcctactattacaggatgatcttcgagaggtacttcccccaggtgagtgactaaatagaacaagttgaaaaacaacacgaatagttcaataccattgtagcaatgttgattgctcaatcttgtttcagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 54 - DNA, 3801 bp, Synthetic construct
  source 1..3801: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccgagcgaccagccgctctacaacgaggacaagtccatcgccgttgccgtcaacggggaggtctacaaccatgaggagcttcgggcacggctctccggacacaggttccggaccggcagtgactgcgaggtcatcgcccatctggtcagtcacccaacacacaccagccttcttcttctacttgatatataaattcaatcgtatgaaacaataagtttggtcaatccgtagcttctagcatacaaaaaaaagatgatccagataatctctatacatcctaatttgaggttaatttcatatagtaatcagcgaaattaaagttaagtgctagaccattatatctctaatttgttgattaaaattttaataaaaggaagatcttggcgaatatacataggtggacatggttccacctgcacctgaatgacaattaaaattttaaaatatatcagagaattaaacaattctgaattttttaaacaaatatgatccggtgttctatatatatccaccgtggactttcatgaaaaaataacatccattgtgtaccgggataaaatgaaaaaaaacaggtgctctaaaatggtttgaaaatgctcggctcgttaagcttaacgacaagaaaactataatcggctcggccgtaagtacttgacatgcatcaccccttttgctccagttactctgtgttttttatgcaagaagaaagtgaccacacatgaggaggtgcactagttattgtctcctatgggttgagttgggtcaattagatagagttgatgggttggtgtgccatgaaatgttgccgcttaacttaagaatatgtgttgtgttgtattaacgagctgagctgctcgtgaaattcgttaactcctcattaagcttattaagtttaacgagctgaaactagcgtgattggccatgagctacgagtttaatgacccgagctactgagcgttcattatgcttgcgagctacgaggttttggtccacccctaaccacgggtgtgatttcttggtgggtaggacactcgaccaggtttgttttaaaaaatcttttttttttgctaattttcctgatttctctaatcatgtgagtgcgtgcacaccagagccagaactccatttttagattcttgcatgattaaaaagaaagtagaactttggaaaggtggagaaatgtacagctcttagtggcattctctttgtttagcatgacctgactattttctcccttttgatatatagtatgaggaatacggagaaagcttcattgacatgttggatggtgttttctccttcgtgttacttgacgcacgagataacagcttcattgctgctcgtgatgccattggtgtcacgcctctctacattggctggggaattgatggtatagtatcaaaagtgcttcaggaatcttttcatgaaacaaaactatcatttgctctgtgttaatatacaccttgtgaagtagtggttgtcattatcaacaacatgcacactgtggaaaattgtcaggttcagtgtggatatcttcggagatgaaaggactaaacgatgattgtgagcacttcgagatcttcccgcctggtaatctttactccagcaaagagaagtccttcaagagatggtataaccctccttggttctctgaggtcatcccctcggttccctatgacccactgcgtctcagatcggcatttgaaaaggtcacaaactatacgcacctcaactaaccattatgtgctagtatctttgaaaacagatatgtaaccaaattaaggagtcatcgccatgattcttctaactgcatcttctgcattcaggctgttatcaagaggctcatgacagatgttccatttggcgtcctcctctccggtggtctcgactcatcattggtggctgctgtcgcagcccgtcatttcgctgggacgaaggctgcaaagcgctggggaactaggctccactccttctgtgtggggcttgaggtctgaaactgaaagggaacatgtgcatgtttattgaagtttcaaagaaataggacttgcatccatgttatcttaatttatattgtagggatcaccagatctcaaggctgcaaaggaggtcgcggatcacctgggtaccgtgcaccacgagttcaacttcacagttcaggtacgatattactactaagaggcataacgatcgtagcttatttgttgatcgcaatacgcaaccagtaatgttaattgcataaattgctataggatggcatcgatgcaattgaagatgtgatataccacattgaaacatatgatgtgacgacgatcagggcaagcacactgatgttccagatgtcacgcaagatcaaggcgcttggagtcaagatggtcatctccggtgagggtgccgatgagatcttcggagggtacttgtatttccacaaggcccctaacaaggaggagttccaccaggaaacatgtcggaaggtatttaaaatgcaaacattgcagaggaacagaaagaataggacttatccataatagcactgtgctctgacatgaattgttctgcagataaaagctctccatcagtacgattgcttgagggccaacaaagcaacatctgcatggggccttgaggttcgtgtgccattcttggacaaggagttcatcaatgaggctatgagcatagatcccgaatggaagatggtaagcacaaaatatatatttcacggggaaagcccaaaacatttcaaaagaatacacgtcgaaaaaacatttcaaacgaatgggaagttggaaagaatggactgcgttgttttctaacttttaatcacacagatccggcctgatcttggaagaattgagaaatggatactgaggaaagcgttcgatgatgaggagcgacccttcctgccgaaggtaacatttttatacacttcagtcagatccagctggagtagtggagttattaacatgttctttttttatacatgtagcatattctgtacaggcagaaggagcagtttagtgatggtgttgggtatagctggattgatggcctgaaggatcatgcagcctcaaatgtaaatgaacaatttttttattttaaagaattctttatgatgtttgttttgacaaaactaatacattccctgatagaaagatgtgaccacaatatctgacctatatgaaatgaaatcactgaatgcaggtgagtgataagatgatgtccaatgcaaagttcatctacccacacaacaccccaacaactaaagaggcctactattacaggatgatctttgagaggtacttcccccaggtgagtgactaaatagaacaggttgaagaacaacatgaatagttcaataccattgtagcaatgttaatggaagcaatgttggttgcttgatcttgtttcagagctcggcgatcctcacggtgccaggcgggccaagcgtggcgtgcagcacagccaaggctatagagtgggatgcccaatggtctgggaacctggacccctctgggagagcagcgcttggagtccatctctcagcctacgagcaggacacggtcgctgtgggaggtagcaacaagcctggggtgatgaacaccgtggtacctggtgttgccattgagacttga

SEQ ID NO: 55 - DNA, 4058 bp, Synthetic construct
  source 1..4058: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgagcgaccagccgctctacaacgaggacaagtccgtctccgtcgccgtgaacggggagatttacaaccatgaggagctccgggcacggctctccggccacaggttccggaccggcagtgactgcgaggtcatcgcccatctggtcagtcacccaccgcacacaccaccattctttcttctgcttgatactacgtatatactagtaaatgagcacgtgctgcatgttgatattagggaggaaattaactgcatgttaatattaggtaggatatcaattacgtgttaattatgaaattataggtaggatatcagttacgtgttaattatgagattaatatagatgttgatatttggtatgctatcaattgcacgctcaacatgttgagagctcatcactagagcaatctaggtcgttgaattgacatggtttgatggccgagattaattgaatctgccaatttgggtctttttatattgaaatataggtatagataaattcaacagtataaaacaataagtttggtgaatccgtagcttctagcacagaaacacaaagataatccggataatctctatacatcctaatttgaggttaatttcgtatattaatcagcgaaataaaagttaagtgctagaccattatatctctaatctgttgattacaattttaataaaaggaagatcttggcgaatatacatcggtgcacctgaatgacgagtaagattttaaaatataacaaagaattaaacaattctgattttttaaacaaatatgatgagtgttctatatccactgtggacttccatgaaaaaatgacatccattgtgtaccgggataaaatgaaaaaaaaacatatgctctaaaatggtttgaaaatgctcggctcgttaagcttaacgacaagaaaactatgatcggctcgtaagtacttgacatgcaataaccccttttgctccagttactgtgtgttttttatgcaagaagaaagtgaccagatgtggaggtacactagttcttgtctcctatgggttgagttgggtcaattagatggagttgatgggttggtgtgccatgaaatgttgccgcttaacttaagaatatgtgttgtgttgtattaacgagctgagctgctcgtgaaattcgttaactcctcattaagctcattaagggaaaattatgctcattaagtttaacgagctgaaactagcggtgattggccctgagctatgctcattaagtttaatgacccgagctactgagcattcattatgcttgcgagctatgaggttttggtccatccctaaccacgggtgtgatttcttggtgggtaggacactcgatcaggtttgtttaaaaaaatcattttttttgctatttttcctgatttctctaatcatgtaagtgcgtgcacaccagagccagaactccatttttagattcttgcatgattaaaaagaaagtagaactttggaaaggtggagaaatgtacagctcttagtggcattctttttgtttagcatgacctgactattttctcccttttgatatatagtatgaggaatacggagaaagattcattgacatgttggatggtgttttctccttcgtgttgcttgacgcacgagataagagcttcattgctgctcgtgatgccattggtgtcacgcctctctacattggctggggaattgatggtatagcatcaaaagtggttgaggaatcttttcatgaagcaaacctatcatttgctctgtgttactaaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggttgcaaactatacccgactcaactaaccacactaatatcttggaagaacagagaagtgaccaaatttaggactcatggtcatgattcttctaaattcatgctcttcattcaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgaggtctggaactgaaaggaacatgtgcatgtttattgaagtttcaaagtaatagcacctgcaaccatgctgttatcttaatttatattgtagggctcaccggatctcaaggctgcaaaggaggtcgcggatcacctgggtaccgtgcaccatgagttcaacttcacagttcaggtacgatattactactaagaggcataacgatcgtagcttatttgttgatcacaatacgcaaccagtaatgttaattgcataaattgctataggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaaggtatttaaagtagaagcgtttcagaggagcagaaagaataggacataaccataataacactctgctctgacataaaattgctctacagattaaagctctccatcagtacgattgcttgagggctaacaaagcaacatctgcatggggccttgaggttcgtgtgccattcttggacaaggagttcatcgatgaggctatgagcatagatcccgaatggaagatggtaagcacaaaaaatatatttcacggggaaagcccaaaacatttcgaaagaatacgcgtcaaaaaaatatttcaaacgaatgggaagttagaaagaatggactgcattgttttctaaatttaatgacacagatccggcctgatcttggaagaattgagaaatggatactgaggaaagcgttcgatgatgaggagcaacccttcctgccgaaggtaacatttttatacacttcagtcagatccagttggagtagtggagttattaacaggttctttttttatacatgtagcatattctgtacaggcagaaggagcagtttagtgatggtgttgggtatagctggattgatggcctgaaggatcatgcagcatcaaatgtaaatgaacaatttttttattttgaagaattctttatgatgtttgtttcgacaaaactaatgcattccttgataggaagatgtgactgcaatatctgacctctatgaaatgaaatcactgaatgcaggtgagtgataagatgatgtccaatgcaaagttcatctacccacacaacaccccaacaactaaagaggcctactattacaggatgatcttcgagaggtacttcccccaggtgagtgactaaatagaacaagttgaaaaacaacacgaatagttcaataccattgtagcaatgttgattgctcaatcttgtttcagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 56 - DNA, 4058 bp, Synthetic construct
  source 1..4058: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgtgcgaccagccgctctacaacgaggacaagtccgtctccgtcgccgtgaacggggagatttacaaccatgaggagctccgggcacggctctccggccacaggttccggaccggcagtgactgcgaggtcatcgcccatctggtcagtcacccaccgcacacaccaccattctttcttctgcttgatactacgtatatactagtaaatgagcacgtgctgcatgttgatattagggaggaaattaactgcatgttaatattaggtaggatatcaattacgtgttaattatgaaattataggtaggatatcagttacgtgttaattatgagattaatatagatgttgatatttggtatgctatcaattgcacgctcaacatgttgagagctcatcactagagcaatctaggtcgttgaattgacatggtttgatggccgagattaattgaatctgccaatttgggtctttttatattgaaatataggtatagataaattcaacagtataaaacaataagtttggtgaatccgtagcttctagcacagaaacacaaagataatccggataatctctatacatcctaatttgaggttaatttcgtatattaatcagcgaaataaaagttaagtgctagaccattatatctctaatctgttgattacaattttaataaaaggaagatcttggcgaatatacatcggtgcacctgaatgacgagtaagattttaaaatataacaaagaattaaacaattctgattttttaaacaaatatgatgagtgttctatatccactgtggacttccatgaaaaaatgacatccattgtgtaccgggataaaatgaaaaaaaaacatatgctctaaaatggtttgaaaatgctcggctcgttaagcttaacgacaagaaaactatgatcggctcgtaagtacttgacatgcaataaccccttttgctccagttactgtgtgttttttatgcaagaagaaagtgaccagatgtggaggtacactagttcttgtctcctatgggttgagttgggtcaattagatggagttgatgggttggtgtgccatgaaatgttgccgcttaacttaagaatatgtgttgtgttgtattaacgagctgagctgctcgtgaaattcgttaactcctcattaagctcattaagggaaaattatgctcattaagtttaacgagctgaaactagcggtgattggccctgagctatgctcattaagtttaatgacccgagctactgagcattcattatgcttgcgagctatgaggttttggtccatccctaaccacgggtgtgatttcttggtgggtaggacactcgatcaggtttgtttaaaaaaatcattttttttgctatttttcctgatttctctaatcatgtaagtgcgtgcacaccagagccagaactccatttttagattcttgcatgattaaaaagaaagtagaactttggaaaggtggagaaatgtacagctcttagtggcattctttttgtttagcatgacctgactattttctcccttttgatatatagtatgaggaatacggagaaagattcattgacatgttggatggtgttttctccttcgtgttgcttgacgcacgagataagagcttcattgctgctcgtgatgccattggtgtcacgcctctctacattggctggggaattgatggtatagcatcaaaagtggttgaggaatcttttcatgaagcaaacctatcatttgctctgtgttactaaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggttgcaaactatacccgactcaactaaccacactaatatcttggaagaacagagaagtgaccaaatttaggactcatggtcatgattcttctaaattcatgctcttcattcaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgaggtctggaactgaaaggaacatgtgcatgtttattgaagtttcaaagtaatagcacctgcaaccatgctgttatcttaatttatattgtagggctcaccggatctcaaggctgcaaaggaggtcgcggatcacctgggtaccgtgcaccatgagttcaacttcacagttcaggtacgatattactactaagaggcataacgatcgtagcttatttgttgatcacaatacgcaaccagtaatgttaattgcataaattgctataggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaaggtatttaaagtagaagcgtttcagaggagcagaaagaataggacataaccataataacactctgctctgacataaaattgctctacagattaaagctctccatcagtacgattgcttgagggctaacaaagcaacatctgcatggggccttgaggttcgtgtgccattcttggacaaggagttcatcgatgaggctatgagcatagatcccgaatggaagatggtaagcacaaaaaatatatttcacggggaaagcccaaaacatttcgaaagaatacgcgtcaaaaaaatatttcaaacgaatgggaagttagaaagaatggactgcattgttttctaaatttaatgacacagatccggcctgatcttggaagaattgagaaatggatactgaggaaagcgttcgatgatgaggagcaacccttcctgccgaaggtaacatttttatacacttcagtcagatccagttggagtagtggagttattaacaggttctttttttatacatgtagcatattctgtacaggcagaaggagcagtttagtgatggtgttgggtatagctggattgatggcctgaaggatcatgcagcatcaaatgtaaatgaacaatttttttattttgaagaattctttatgatgtttgtttcgacaaaactaatgcattccttgataggaagatgtgactgcaatatctgacctctatgaaatgaaatcactgaatgcaggtgagtgataagatgatgtccaatgcaaagttcatctacccacacaacaccccaacaactaaagaggcctactattacaggatgatcttcgagaggtacttcccccaggtgagtgactaaatagaacaagttgaaaaacaacacgaatagttcaataccattgtagcaatgttgattgctcaatcttgtttcagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 57 - DNA, 2820 bp, Synthetic construct
  source 1..2820: mol_type = other DNA
atgtgtggcatactggccgtgctggggtgcggcgacgagtcgcaggggaagagggtccacgtcctcgagctctcgcgcaggctcaagcaccgcggcccggactggagcggcctgcaccaggtcgccgacaactacctctgccaccagcgcctcgccatcatcgacccggcctccgagcgaccagccgctctacaacgaggacaagtccgtcgccgtcgccgtcaacggggagatctacaaccatgaggagctccgggcacggctctccggccacagcttccggaccggcagtgactgcgaggtcatcgcccatttggtcagtcacccaccacacacacaccaacattcttcttctgcttgacatattaagtacttcactacaaatctgacctcaaattatttttaccttttaccatggcaaattgaactttttttagtgccattttttttgtttagcatgacctgactattttctcccttttgacatatagtatgaggaatacggagaaagctccattgagatgttggatggtgttttctccttcgtgttgcttgacgcaagagatcacagcttcatcgctgctcgtgatgccattggtgtcacgcctctctacattggctggggaattgatggtataatatcaaaagtgctttcatgaaacagaactatcatttgcactgtgttaatatacaccttgtgaagtagtggctgtcatcatcatcaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcaattccctatgacccactgcgtctcagatcggcatttgaaaaggtcacaaaccacacttgcctcaactaaccgttatgtgcatctttgaaaaacagagatgtaaccaaatttaggagtcaagagtcaactaaccatgattcttctgactgcatcttctgcatccaggctgtcatcaagaggctcatgacagatgttccatttggcgtcctcctctccggtggtctcgactcatcactggtggcagctgtcgcagcccgtcattttgcagggacaaaggctgcaaaacgttggggaactaggctccactccttctgtgttgggcttgaggtctgaaacttaaaggaacatgtgcatgtttatcgaagtttcaaagtaataccacttgcaaccatgctgttatcttaatttatattgtagggatcaccggatctcaaggctgcaaaggaggtcgcggatcatctgggtactgtgcaccatgagttcaacttcacagttcaggcatgatattaccactaagaagcataacgatcgtcgcttatttgttgatcacaatacagaatcactaattttaatagcacatattgctataggacggcatcgatgcaattgaagatgtgatatatcacattgaaacatatgacgtgacgacgatcagggcaagcacactgatgttccagatgtcacgcaagatcaaggcgcttggtgtcaagatggtcatctctggtgagggcgccgatgaaatctttggagggtacttgtatttccacaaggcccctaacaaggaggagttccaccaggaaacatgtcggaaggtctttaagatacaaacgtttcagaggagcagaaacaacatataggaccaacacataacactctgctctgacataaattgttctgcagatcaaagctctccatcagtacgattgcttgagggccaacaaagcaacatctgcatggggccttgaggttcgtgtgccattcttggacaaggagttcatcaatgaggctatgagcatagaccccgaatggaagatggtaagcacaaaacatttcatgagaatgggaaattgaaaatagtgcactgcactgttttctaaatttaatcacacagatccggcctgatcttggaagaattgaaaaatggatattgaggaaagcatttgatgacgaggagaaaccgttcctgccgaaggtaacattttttagactcagcagtcagatccagctggagtagtggagttattaacatgttcttttgtgtgatctatgtagcacattctgtacaggcagaaggagcagtttagtgatggtgttgggtatagctggattgatggcctgaaggatcatgcggcatcaaatgtaaatgaacaattttattcattttaaagaatctttacgatgtttgtttcgacaaaactaatgcattccttgaaagaaagacgcgaccacaatatctgacctatatgacatgaaatcactgaatgcaggtgagtgataagatgatgtccaatgcaaagttcatctacccacacaacaccccaacgactaaagaggcttactattacaggatgatctttgagaggtacttcccccaggtgagtcaataaatagaacaggttgaagaaaacaacatgaatagttcaataccattgtagcaatgttaatggaagcaatgtcggttgctcgatcttgtttcagagctcggcgatcctcacggtgccaggcgggccaagcgtggcgtgcagcacagccaaggctatagagtgggatgcccagtggtctgggaacctggacccctccgggagagcagcgcttggagtccatctctcggcctacgagcaggacacggtagcagtgggaggtagcaacaagcctggggtgatgaggaccgcggtgcctggtgttgccatcgagacctga

SEQ ID NO: 58 - DNA, 4058 bp, Synthetic construct
  source 1..4058: mol_type = other DNA
atgtgcggcatactggcggtgctggggtgcggcgacgagtcgcaggggaagagggtccacgtcctcgagctctctcgcaggctcaagcaccgcggcccggactggagcggcctgcaccaggtcgccgacaactacctatgccaccagcgcctggccatcatcgacccggcctccgcgcgaccagccgctctacaacgaggacaagtccgtctccgtcgccgtgaacggggagatttacaaccatgaggagctccgggcacggctctccggccacaggttccggaccggcagtgactgcgaggtcatcgcccatctggtcagtcacccaccgcacacaccaccattctttcttctgcttgatactacgtatatactagtaaatgagcacgtgctgcatgttgatattagggaggaaattaactgcatgttaatattaggtaggatatcaattacgtgttaattatgaaattataggtaggatatcagttacgtgttaattatgagattaatatagatgttgatatttggtatgctatcaattgcacgctcaacatgttgagagctcatcactagagcaatctaggtcgttgaattgacatggtttgatggccgagattaattgaatctgccaatttgggtctttttatattgaaatataggtatagataaattcaacagtataaaacaataagtttggtgaatccgtagcttctagcacagaaacacaaagataatccggataatctctatacatcctaatttgaggttaatttcgtatattaatcagcgaaataaaagttaagtgctagaccattatatctctaatctgttgattacaattttaataaaaggaagatcttggcgaatatacatcggtgcacctgaatgacgagtaagattttaaaatataacaaagaattaaacaattctgattttttaaacaaatatgatgagtgttctatatccactgtggacttccatgaaaaaatgacatccattgtgtaccgggataaaatgaaaaaaaaacatatgctctaaaatggtttgaaaatgctcggctcgttaagcttaacgacaagaaaactatgatcggctcgtaagtacttgacatgcaataaccccttttgctccagttactgtgtgttttttatgcaagaagaaagtgaccagatgtggaggtacactagttcttgtctcctatgggttgagttgggtcaattagatggagttgatgggttggtgtgccatgaaatgttgccgcttaacttaagaatatgtgttgtgttgtattaacgagctgagctgctcgtgaaattcgttaactcctcattaagctcattaagggaaaattatgctcattaagtttaacgagctgaaactagcggtgattggccctgagctatgctcattaagtttaatgacccgagctactgagcattcattatgcttgcgagctatgaggttttggtccatccctaaccacgggtgtgatttcttggtgggtaggacactcgatcaggtttgtttaaaaaaatcattttttttgctatttttcctgatttctctaatcatgtaagtgcgtgcacaccagagccagaactccatttttagattcttgcatgattaaaaagaaagtagaactttggaaaggtggagaaatgtacagctcttagtggcattctttttgtttagcatgacctgactattttctcccttttgatatatagtatgaggaatacggagaaagattcattgacatgttggatggtgttttctccttcgtgttgcttgacgcacgagataagagcttcattgctgctcgtgatgccattggtgtcacgcctctctacattggctggggaattgatggtatagcatcaaaagtggttgaggaatcttttcatgaagcaaacctatcatttgctctgtgttactaaacatgcacatctctgtaaaattgtcaggttcagtgtggatatcttcggagatgaaaggactaaacgatgattgtgagcacttcgagatcttcccgcctggtaatctttactccagcaaagaaaagtgcttcaagagatggtataaccctccttggttctctgaggtcatcccctcggttccctatgacccactgcgtctcagatcggcatttgaaaaggttgcaaactatacccgactcaactaaccacactaatatcttggaagaacagagaagtgaccaaatttaggactcatggtcatgattcttctaaattcatgctcttcattcaggctgtaatcaagaggctcatgacagatgttccatttggtgtcctcctctccggtggcctcgactcatcactggtggcggctgtcgcagcccgtcatttcgcagggacaaaggctgcaaagcgctggggaactaggctccactccttctgtgttgggcttgaggtctggaactgaaaggaacatgtgcatgtttattgaagtttcaaagtaatagcacctgcaaccatgctgttatcttaatttatattgtagggctcaccggatctcaaggctgcaaaggaggtcgcggatcacctgggtaccgtgcaccatgagttcaacttcacagttcaggtacgatattactactaagaggcataacgatcgtagcttatttgttgatcacaatacgcaaccagtaatgttaattgcataaattgctataggacggcatcgatgcaattgaagatgtgatatatcacattgagacatatgatgtgacgacgatcagggcaagcacattgatgttccagatgtcacgcaagatcaaggcgcttggtgtcaagatggtcatctctggtgagggtgccgacgaaatctttggagggtacttgtatttccacaaggcccctaacaaggaggagttccaccaggaaacatgtcggaaggtatttaaagtagaagcgtttcagaggagcagaaagaataggacataaccataataacactctgctctgacataaaattgctctacagattaaagctctccatcagtacgattgcttgagggctaacaaagcaacatctgcatggggccttgaggttcgtgtgccattcttggacaaggagttcatcgatgaggctatgagcatagatcccgaatggaagatggtaagcacaaaaaatatatttcacggggaaagcccaaaacatttcgaaagaatacgcgtcaaaaaaatatttcaaacgaatgggaagttagaaagaatggactgcattgttttctaaatttaatgacacagatccggcctgatcttggaagaattgagaaatggatactgaggaaagcgttcgatgatgaggagcaacccttcctgccgaaggtaacatttttatacacttcagtcagatccagttggagtagtggagttattaacaggttctttttttatacatgtagcatattctgtacaggcagaaggagcagtttagtgatggtgttgggtatagctggattgatggcctgaaggatcatgcagcatcaaatgtaaatgaacaatttttttattttgaagaattctttatgatgtttgtttcgacaaaactaatgcattccttgataggaagatgtgactgcaatatctgacctctatgaaatgaaatcactgaatgcaggtgagtgataagatgatgtccaatgcaaagttcatctacccacacaacaccccaacaactaaagaggcctactattacaggatgatcttcgagaggtacttcccccaggtgagtgactaaatagaacaagttgaaaaacaacacgaatagttcaataccattgtagcaatgttgattgctcaatcttgtttcagagctcggccatcctgacggtgccaggcgggccaagcgtggcgtgcagcacagccaaggctatagagtgggatgcccagtggtctggaaacctggacccctctgggagagcagcgcttggagtccatctctcagcctacgagcaggacacagtcgctgtgggaggtagcaacaagcatggggtgatgaagaccgtggtgcctggtgttgccattgagacctga

SEQ ID NO: 59 - DNA, 3800 bp, Synthetic construct
  source 1..3800: mol_type = other DNA
atgtgcggcatactagcggtgctggggtgcggcgacgagtcgcaggggaagagggtccacgtgctagagctctcgcgcaggctcaagcaccggggcccggactggagcggcctgcaccaggtcgccgacaactacctctgccaccagcgcctcgccatcatcgacccggcctccggcgaccagccgctctacaacgaggacaagtccatcgccgttgccgtcaacggggaggtctacaaccatgaggagcttcgggcacggctctccggacacaggttccggaccggcagtgactgcgaggtcatcgcccatctggtcagtcacccaacacacaccagccttcttcttctacttgatatataaattcaatcgtatgaaacaataagtttggtcaatccgtagcttctagcatacaaaaaaaagatgatccagataatctctatacatcctaatttgaggttaatttcatatagtaatcagcgaaattaaagttaagtgctagaccattatatctctaatttgttgattaaaattttaataaaaggaagatcttggcgaatatacataggtggacatggttccacctgcacctgaatgacaattaaaattttaaaatatatcagagaattaaacaattctgaattttttaaacaaatatgatccggtgttctatatatatccaccgtggactttcatgaaaaaataacatccattgtgtaccgggataaaatgaaaaaaaacaggtgctctaaaatggtttgaaaatgctcggctcgttaagcttaacgacaagaaaactataatcggctcggccgtaagtacttgacatgcatcaccccttttgctccagttactctgtgttttttatgcaagaagaaagtgaccacacatgaggaggtgcactagttattgtctcctatgggttgagttgggtcaattagatagagttgatgggttggtgtgccatgaaatgttgccgcttaacttaagaatatgtgttgtgttgtattaacgagctgagctgctcgtgaaattcgttaactcctcattaagcttattaagtttaacgagctgaaactagcgtgattggccatgagctacgagtttaatgacccgagctactgagcgttcattatgcttgcgagctacgaggttttggtccacccctaaccacgggtgtgatttcttggtgggtaggacactcgaccaggtttgttttaaaaaatcttttttttttgctaattttcctgatttctctaatcatgtgagtgcgtgcacaccagagccagaactccatttttagattcttgcatgattaaaaagaaagtagaactttggaaaggtggagaaatgtacagctcttagtggcattctctttgtttagcatgacctgactattttctcccttttgatatatagtatgaggaatacggagaaagcttcattgacatgttggatggtgttttctccttcgtgttacttgacgcacgagataacagcttcattgctgctcgtgatgccattggtgtcacgcctctctacattggctggggaattgatggtatagtatcaaaagtgcttcaggaatcttttcatgaaacaaaactatcatttgctctgtgttaatatacaccttgtgaagtagtggttgtcattatcaacaacatgcacactgtggaaaattgtcaggttcagtgtggatatcttcggagatgaaaggactaaacgatgattgtgagcacttcgagatcttcccgcctggtaatctttactccagcaaagagaagtccttcaagagatggtataaccctccttgattctctgaggtcatcccctcggttccctatgacccactgcgtctcagatcggcatttgaaaaggtcacaaactatacgcacctcaactaaccattatgtgctagtatctttgaaaacagatatgtaaccaaattaaggagtcatcgccatgattcttctaactgcatcttctgcattcaggctgttatcaagaggctcatgacagatgttccatttggcgtcctcctctccggtggtctcgactcatcattggtggctgctgtcgcagcccgtcatttcgctgggacgaaggctgcaaagcgctggggaactaggctccactccttctgtgtggggcttgaggtctgaaactgaaagggaacatgtgcatgtttattgaagtttcaaagaaataggacttgcatccatgttatcttaatttatattgtagggatcaccagatctcaaggctgcaaaggaggtcgcggatcacctgggtaccgtgcaccacgagttcaacttcacagttcaggtacgatattactactaagaggcataacgatcgtagcttatttgttgatcgcaatacgcaaccagtaatgttaattgcataaattgctataggatggcatcgatgcaattgaagatgtgatataccacattgaaacatatgatgtgacgacgatcagggcaagcacactgatgttccagatgtcacgcaagatcaaggcgcttggagtcaagatggtcatctccggtgagggtgccgatgagatcttcggagggtacttgtatttccacaaggcccctaacaaggaggagttccaccaggaaacatgtcggaaggtatttaaaatgcaaacattgcagaggaacagaaagaataggacttatccataatagcactgtgctctgacatgaattgttctgcagataaaagctctccatcagtacgattgcttgagggccaacaaagcaacatctgcatggggccttgaggttcgtgtgccattcttggacaaggagttcatcaatgaggctatgagcatagatcccgaatggaagatggtaagcacaaaatatatatttcacggggaaagcccaaaacatttcaaaagaatacacgtcgaaaaaacatttcaaacgaatgggaagttggaaagaatggactgcgttgttttctaacttttaatcacacagatccggcctgatcttggaagaattgagaaatggatactgaggaaagcgttcgatgatgaggagcgacccttcctgccgaaggtaacatttttatacacttcagtcagatccagctggagtagtggagttattaacatgttctttttttatacatgtagcatattctgtacaggcagaaggagcagtttagtgatggtgttgggtatagctggattgatggcctgaaggatcatgcagcctcaaatgtaaatgaacaatttttttattttaaagaattctttatgatgtttgttttgacaaaactaatacattccctgatagaaagatgtgaccacaatatctgacctatatgaaatgaaatcactgaatgcaggtgagtgataagatgatgtccaatgcaaagttcatctacccacacaacaccccaacaactaaagaggcctactattacaggatgatctttgagaggtacttcccccaggtgagtgactaaatagaacaggttgaagaacaacatgaatagttcaataccattgtagcaatgttaatggaagcaatgttggttgcttgatcttgtttcagagctcggcgatcctcacggtgccaggcgggccaagcgtggcgtgcagcacagccaaggctatagagtgggatgcccaatggtctgggaacctggacccctctgggagagcagcgcttggagtccatctctcagcctacgagcaggacacggtcgctgtgggaggtagcaacaagcctggggtgatgaacaccgtggtacctggtgttgccattgagacttga

SEQ ID NO: 60 - DNA, 74 bp, Synthetic construct
  source 1..74: mol_type = other DNA
atcaacaacatgcacactgtggaaaattgtcaggttcagtgtgratatcttcggagatgaaaggactaaacgat

SEQ ID NO: 61 - DNA, 74 bp, Synthetic construct
  source 1..74: mol_type = other DNA
tagttgttgtacgttggacaccttttaacagtaccagtcacacytatagaagcctctactttcctgatttgcta

SEQ ID NO: 62 - DNA, 24 bp, Synthetic construct
  source 1..24: mol_type = other DNA
ctatagaagcctctactttcctga

SEQ ID NO: 63 - DNA, 24 bp, Synthetic construct
  source 1..24: mol_type = other DNA
ttatagaagcctctactttcctga

SEQ ID NO: 64 - DNA, 79 bp, Synthetic construct
  source 1..79: mol_type = other DNA
gtgtcacgcctctctacattggctrgggaattgatggtatagtatagtatcaaaagtgcttcaggaatcttttcatgaa

SEQ ID NO: 65 - DNA, 74 bp, Synthetic construct
  source 1..74: mol_type = other DNA
cacagtgcggagagatgtaaccgaycccttaactaccatatcatagttttcacgaagtccttagaaaagtactt

SEQ ID NO: 66 - DNA, 20 bp, Synthetic construct
  source 1..20: mol_type = other DNA
acgcctctctacattggcta

SEQ ID NO: 67 - DNA, 20 bp, Synthetic construct
  source 1..20: mol_type = other DNA
acgcctctctacattggctg

SEQ ID NO: 68 - DNA, 56 bp, Triticum aestivum
  source 1..56: mol_type = genomic DNA
catcatcgacccggcctccggcgaccagccgctctacaacgaggacaagtccatcg

SEQ ID NO: 69 - DNA, 56 bp, Triticum aestivum
  source 1..56: mol_type = genomic DNA
catcatcgacccggcctccggcgaccagccgctctacaacgaggacaagtccgtcg

SEQ ID NO: 70 - DNA, 56 bp, Triticum aestivum
  source 1..56: mol_type = genomic DNA
catcatcgacccggcctccggcgaccagccgctctacaacgaggacaagtccgtct

SEQ ID NO: 71 - DNA, 56 bp, Triticum aestivum
  source 1..56: mol_type = genomic DNA
catcatcgaccctgcctctggcgaccagcccctctacaacgaggacaagtccatcg

SEQ ID NO: 72 - DNA, 56 bp, Triticum aestivum
  source 1..56: mol_type = genomic DNA
catcatcgaccctgcctctggcgaccagccgctctacaacgaggacaagtccatcg

SEQ ID NO: 73 - DNA, 56 bp, Triticum aestivum
  source 1..56: mol_type = genomic DNA
cattatcgaccctgcctctggcgaccagccgctctacaacgaggacaagtccatcg

SEQ ID NO: 74 - DNA, 56 bp, Triticum aestivum
  source 1..56: mol_type = genomic DNA
cattgttgatcccacatctggagaccagccattgtacaacgaggacaaaacagttg

SEQ ID NO: 75 - DNA, 56 bp, Triticum aestivum
  source 1..56: mol_type = genomic DNA
tattgttgatcccacatctggagaccagccattgtacaacgaggacaaaacagttg

SEQ ID NO: 76 - DNA, 56 bp, Triticum aestivum
  source 1..56: mol_type = genomic DNA
tattgttgatcctacatctggagaccagccactgtacaacgaggacaaaacagttg

SEQ ID NO: 77 - DNA, 56 bp, Triticum aestivum
  source 1..56: mol_type = genomic DNA
tattgttgatcctacatctggagaccagccattgtacaacgaggacaaaacagttg

SEQ ID NO: 78 - DNA, 56 bp, Triticum aestivum
  source 1..56: mol_type = genomic DNA
cgtcgtctccccgctctccggcgaccagcccctctacaacgaggaccggaccgtcg

SEQ ID NO: 79 - DNA, 56 bp, Triticum aestivum
  source 1..56: mol_type = genomic DNA
cgtcgtctccccgctctccggtgaccagcccctctacaacgaggaccggaccgtcg

SEQ ID NO: 80 - DNA, 30 bp, Triticum aestivum
  source 1..30: mol_type = genomic DNA
cccggcctccggcgaccagccgctctacaa

SEQ ID NO: 81 - DNA, 29 bp, Synthetic construct
  source 1..29: mol_type = other DNA
cccggcctccgcgaccagccgctctacaa

SEQ ID NO: 82 - DNA, 31 bp, Synthetic construct
  source 1..31: mol_type = other DNA
cccggcctccgggcgaccagccgctctacaa

SEQ ID NO: 83 - DNA, 31 bp, Synthetic construct
  source 1..31: mol_type = other DNA
cccggcctccgagcgaccagccgctctacaa